(19) United States
(12) Patent Application Publication
(10) Pub. No.: US 2019/0321494 A1
(43) Pub. Date: Oct. 24, 2019

(54) POLYMALIC ACID BASED NANOCONJUGATES FOR IMAGING

(71) Applicant: Cedars-Sinai Medical Center, Los Angeles, CA (US)

(72) Inventors: Keith L. Black, Los Angeles, CA (US); Julia Ljubimova, Studio City, CA (US); Alexander Ljubimov, Studio City, CA (US); Eggehard Holler, Los Angeles, CA (US)

(73) Assignee: Cedars-Sinai Medical Center, Moscow (RU)

(21) Appl. No.: 16/434,545

(22) Filed: Jun. 7, 2019

Related U.S. Application Data
(63) Continuation-in-part of application No. 13/441,599, filed on Apr. 6, 2012, now Pat. No. 10,383,958.
(60) Provisional application No. 61/472,362, filed on Apr. 6, 2011.

(51) Int. Cl. A61K 49/16; A61K 49/08; A61K 49/10; C07K 16/22; C07K 16/32

(52) U.S. Cl. CPC A61K 49/16; A61K 49/085; C07K 2317/24; C07K 16/22; C07K 16/32; A61K 49/108

(57) ABSTRACT

Nanoconjugates that include a polymalic-based molecular scaffold with one or more imaging moiety and one or more targeting modules attached to the scaffold are provided. Methods of targeting a diseased cell or a diseased tissue in a subject by administering the nanoconjugate are described. Methods of synthesizing the nanoconjugate are also provided.

This application is a continuation-in-part of U.S. patent application Ser. No. 13/441,599, filed on Apr. 6, 2015, which claims the benefit of U.S. Provisional Application No. 61/472,362, filed Apr. 6, 2011, both of which are incorporated by reference as if fully set forth.

### STATEMENT REGARDING FEDERALLY SPONSORED RESEARCH OR DEVELOPMENT

The invention was made with government support under Grant Nos. CA123495 and CA151815 awarded by National Institutes of Health. The government has certain rights in the invention.

### FIELD OF INVENTION

The disclosure relates to nanoconjugates containing imaging moieties and targeting modules attached to a polymalic acid-based molecular scaffold. The disclosure also relates to methods of synthesizing nanoconjugates and targeting the diseased cells or tissues in by administering nanoconjugates to a subject.

### BACKGROUND

Diagnostic imaging allows avoidance of unnecessary invasive surgical interventions by confirmation of the nature of various pathological conditions including differentiating between edema and a tumor, detection of multiple metastases, or detection of mental illness or dementia. Non-invasive imaging may be especially useful for diagnostics of diseases or pathological conditions of the human brain, which is not easily accessible by many conventional probing methods such as biopsy and light imaging. Non-invasive imaging is also needed for diagnosis of Alzheimer disease (AD), the most common form of dementia observed in people over 65 years of age.

The oldest approach to diagnose the AD was demonstration of Alzheimer's plaques in human tissue post mortem by employing small chemical compounds that attached specifically to the plaques and that could be visualized by staining ex vivo or by radioactive scintigram in vivo (Newberg A B et al. 2006 J Nuc Med 47:748).

After mouse models became available for AD and cancers, such as triple negative breast cancer, HER2-positive breast cancer, and glioblastoma, in vivo imaging methods could be developed. In vivo imaging approaches utilized fluorescent agents or tagged antibodies binding specifically to components of the diseased cells or tissues, or employed positron emission tomography (PET; Raymond S B et al. 2008 Plos One 3:e2175, 1; Klunk W E et al. 2004 Annals Neurol 55:306).

Although some of these approaches could demonstrate the existence of the diseased tissues, applications required long exposure times and were of insufficient resolution for clearly distinguishing details, or small Alzheimer's plaques. Breakthrough imaging techniques made use of magnetic resonance imaging (MRI). MRI is one of the most advanced non-invasive imaging systems due to application of high resolution contrast agents that include gadolinium (Gd). However, MRI fails to differentiate pathological conditions occurring within a brain. For example, MRI cannot distinguish cancer types, or even cancer from other malignancies. An inefficiency of many in vivo imaging approaches, including MRI, stems from the inability of the contrasting agents, such as gadolinium, to cross the blood-brain barrier (BBB) in combination with rapid elimination of the contrast agent through the kidneys.

### SUMMARY

In an aspect, the invention relates to a nanoconjugate that includes a polymalic acid-based molecular scaffold, at least one imaging moiety and at least one targeting module. One or more of the at least one imaging moiety and one or more of the at least one targeting module is conjugated to the polymalic-acid based molecular scaffold.

In an aspect, the invention relates to a kit for facilitating imaging of a cell or a tissue in a subject. The kit contains a nanoconjugate that includes a polymalic acid-based molecular scaffold, at least one imaging moiety and at least one targeting module. One or more of the at least one imaging moiety and one or more of the at least one targeting module is conjugated to the polymalic-acid based molecular scaffold.

In an aspect, the invention relates to a method of targeting a cell or a tissue in a subject. The method includes administering to the subject a composition that includes a polymalic acid-based molecular scaffold, at least one imaging moiety and at least one targeting module. One or more of the at least one imaging moiety and one or more of the at least one targeting module is conjugated to the polymalic-acid based molecular scaffold.

In an aspect, the invention relates to a method of synthesizing a nanoconjugate. The method involves providing a polymalic acid having a plurality of pendant carboxyl groups. The method further involves reacting a compound containing sulfhydryl groups and amino acid groups through the pendant carboxyl groups to add sulfhydryl groups to the polymalic acid to form an activated polymalic acid. The method involves reacting at least one imaging moiety containing a sulfhydryl binding group to the activated polymalic acid to form a preconjugate. The method also involves reacting at least one targeting module containing a sulfhydryl binding group to the preconjugate.

In an aspect, the invention relates to a method of imaging a cell or a tissue in a brain of a subject and detecting cancer The method involves co-administering to a subject a contrast agent and a composition comprising a polymalic acid-based molecular scaffold conjugated to at least one imaging moiety, and at least one targeting module. The imaging moiety comprises a chelating molecule and a paramagnetic ion chelated by the chelating molecule, and the targeting module is independently selected from the group consisting of: an antibody, a peptide, a polypeptide, an oligonucleotide, and a therapeutic chemical. Co-administering comprises sequential administering of the contrast agent and the composition. The method involves visualizing the contrast agent and the imaging moiety of the composition in the brain of the subject by an MRI technique. The method also involves detecting the presence of a cancer in the brain of the subject. Detecting comprises obtaining a first MRI image of the brain after administering the contrast agent, obtaining a second MRI image of the brain after administering the composition, measuring the intensity of an MRI signal in the first image and the second image; and comparing the intensity and location of the MRI signal in the first image with the intensity and location of the MRI signal in the second MRI image. The similarity in the intensity and location of the MRI signal in the first MRI image relative to the second MRI image is indicative of the presence of the cancer in the brain of the subject.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The following detailed description of the preferred embodiments will be better understood when read in conjunction with the appended drawings. For the purpose of illustration, there are shown in the drawings embodiments which are presently preferred. It is understood, however, that the invention is not limited to the precise arrangements and instrumentalities shown. In the drawings:

FIG. 1 is a schematic drawing illustrating a nanoconjugate designed to facilitate imaging of triple negative breast cancer (TNBC) metastasized to brain.

FIG. 2 is a diagram illustrating synthesis of gadolinium (Gd)-1,4,7,10-tetraazocyclododecane-1,4,7,10-tetraacetic acid (DOTA) amine.

FIG. 3 is a diagram illustrating synthesis of the Gd-DOTA-Polycefin nanoconjugate.

FIG. 4 illustrates the HPLC elution profile of the Gd-DOTA-Polycefin nanoconjugate containing Cetuximab.

FIG. 5 is a set of line graphs illustrating calculation of T1-relaxivity for a Polycefin nanoconjugate that includes polymalic acid (P), 12% Gd-DOTA and 15% 2-mercapto-ethane-1-amine (MEA).

FIG. 6 is a set of line graphs illustrating affinity determination of monoclonal antibody specific to mouse transferrin receptor (anti-MsTfR mAb) by saturation ELISA. Solid line indicates free anti-MsTfR mAb. Broken line indicates MsTfR mAb attached to the Gd-DOTA-Polycefin nanoconjugate that also contains Cetuximab and AlexaFluor 680.

FIG. 7 is a set of Fluorescence Activated Cell Sorting (FACS) histograms illustrating binding of a Rhodamine-labelled Gd-DOTA-Polycefin nanoconjugate containing Cetuximab to an epidermal growth factor receptor (EGFR) expressed in MDA-MB-468 cells in comparison to free Cetuximab and phosphate buffered saline (PBS).

FIG. 8 is a set of MRI images showing brain sections of mice having TNBC metastatic tumors. Images on the left were obtained without a contrast agent administered to animals. Images on the right were obtained after animals received a Polycefin-Gd nanoconjugate intravenously. Scale bar=50 μm.

FIG. 9 is a set of MRI images showing tumors in brain sections of mice having TNBC metastatic tumors. Top images were taken 15 minutes (left) and 1 hour 45 minutes (right) after administering commercially available Gd(III) enhancer reagent to animals. Bottom images were taken 15 minutes (left) and 3 hours 15 minutes (right) after administering to animals a Polycefin nanoconjugate containing polymalic acid, Gd-DOTA, MsTfR, Cetuximab and Alexa Fluor 680 dye. Scale bar=50 μm.

FIGS. 10A and 10B illustrate Xenogen fluorescence imaging of animals injected with a Polycefin-Gd-DOTA nanoconjugate containing Gd-DOTA, MsTfR, Cetuximab Alexa Fluor 680 dye.

FIG. 11 is a set of line graphs illustrating MRI kinetics for tumors after injecting to animals clinically used Gd (III) (open circles) and a Polycefin nanoconjugate containing Gd-DOTA, MsTfR, Cetuximab and Alexa Fluor 680 (closed circles).

FIGS. 12A and 12B illustrate MRI kinetics for parts of the brain having tumor (solid line) in comparison with healthy parts of the brain (broken line) after injecting to the subjects clinically used Gd(III) (FIG. 12A) and a Gd-DOTA-Polycefin nanoconjugate containing Gd-DOTA, MsTfR, Cetuximab and Alexa Fluor 680 (FIG. 12B).

FIGS. 13A to 13D are a set of schematic drawings illustrating nanoconjugates designed to target primary brain and TNBC metastasized to brain (FIG. 13A), and HER2 positive breast cancer metastasized to brain (FIG. 13B) glioblastoma (FIG. 13C), in comparison to a control molecule lacking specific targeting modules (FIG. 13D).

FIG. 14 is a schematic drawing illustrating a nanoconjugate designed to facilitate imaging of Alzheimer's plaques.

FIG. 15 is a diagram illustrating synthesis of a curcumin-PEG₁₀₀₀-amine.

FIG. 16 is a diagram illustrating attachment of curcumin and Gd-DOTA modules to polymalic acid.

FIG. 17 is a set of photographs of fluorescent microscopy of slices of human brain having AD (top images) and normal (bottom images) stained with 20 μM of free curcumin (right) and 20 μM of a Polycefin-curcumin nanoconjugate (left).

### DETAILED DESCRIPTION OF THE PREFERRED EMBODIMENTS

Certain terminology is used in the following description for convenience only and is not limiting. The words “right,” “left,” “top,” and “bottom” designate directions in the drawings to which reference is made.

The words “a” and “one,” as used in the claims and in the corresponding portions of the specification, are defined as including one or more of the referenced item unless specifically stated otherwise. This terminology includes the words above specifically mentioned, derivatives thereof, and words of similar import. The phrase “at least one” followed by a list of two or more items, such as “A, B, or C,” means any individual one of A, B or C as well as any combination thereof.

An embodiment provides a nanoconjugate that may include a polymalic acid-based molecular scaffold, one or more imaging moieties and one or more targeting modules. At least one of the imaging moieties and at least one of the targeting modules may be conjugated to the polymalic acid-based molecular scaffold. All of the imaging moieties may be conjugated to the polymalic acid-based molecular scaffold. All of the targeting modules may be conjugated to the polymalic acid-based molecular scaffold.

Conjugated means covalently bound.

In an embodiment, the nanoconjugate may be Polycefin. As used herein, the term “Polycefin” refers to a family of compounds based on a polymalic acid as the platform for attachment of various specific residues for therapeutic targeting. Polycefin may include polymalic acid derived from a slime mold. Polycefin may be 20 to 30 nm in size and may act as a drug. Polycefin may be engineered to transport other therapeutic molecules. The polymalic acid (PMLA) may include a homopolymer that contains a main chain ester linkage. The polymalic acid may be obtained from cultures of Physarum polycefallum. The polymalic acid may be of any length and of any molecular mass. The polymalic acid may have a molecular mass of 10, 20, 30, 40, 50, 60, 70, 80, 90, 95, or 100 kDa, or more. The polymalic acid may have a molecular mass in a range between any two of the following molecular masses: 10, 20, 30, 40, 50, 60, 70, 80, 90, 95, or 100 kDa. The polymalic acid may be at least one of biodegradable and of a high molecular flexibility, soluble in water (when ionized) and organic solvents (in its acid form), non-toxic, or non-immunogenic (Lee Bs et al., Water-soluable aliphatic polyesters: poly(malic acid)s, in: Biopolymers, vol. 3a (Doi Y, Steinbuchel A eds., pp 75-103, Wiley-VCH, New York 2002, which is incorporated herein by reference as if fully set forth).

In an embodiment, a polymalic acid may be used as a molecular scaffold carrying target modules. In an embodiment, targeting modules may have functions in addition to targeting. Polymalic acid-based molecular scaffolds that may be in embodiments herein were described in PCT Appl. Nos. PCT/US04/40660, filed Dec. 3, 2004, PCT/US09/40252, filed Apr. 10, 2009, and PCT/US10/59919, filed Dec. 10, 2010, PCT/US10/62515, filed Dec. 30, 2010; and U.S. application Ser. No. 10/580,999, filed Mar. 12, 2007, issued as U.S. Pat. No. 7,935,677, and Ser. No. 12/935,110, filed Sep. 28, 2010. All of the foregoing PCT and U.S. applications are incorporated herein by reference as if fully set forth.

A polymalic acid-based molecular scaffold may be a molecule having at least two or more targeting modules attached to the polymalic acid-based molecular scaffold. The targeting modules may also transport a drug, or other therapeutic entity to a targeted tissue.

In an embodiment, the polymalic acid-based molecular scaffold may be based on poly(β-L-malic acid). The poly(β-L-malic acid) may be chemically conjugated at its carboxylic groups at defined ratios with a variety of modules.

In an embodiment, the nanoconjugate having a polymalic acid-based molecular scaffold may target cells or tissues with high specificity. The high specificity of nanoconjugates as drug vehicles may result from enhanced permeability and retention in target tissues that originates from high molecular mass, which may be greater than 20000 (Duncan R. 1999 Research Focus 2:441; Seymour L W et al., 1995 Eur J Cancer Res 31A:766).

In an embodiment, the one or more imaging moieties may include a compound suitable to facilitate an imaging procedure. The compound may be a contrast agent. An imaging may be any imaging procedure used as a clinical diagnostic tool. An imaging may be an MRI procedure that allows non-invasive imaging of optically opaque subjects and may provide contrast among soft tissues at high spatial resolution. An imaging moiety in the one or more imaging moieties may be a chelating molecule used for MRI. The chelating molecule may be but is not limited to 1,4,7,10-tetraazocyclododecane-1,4,7,10-tetraacetic acid (DOTA), dibenzo-DOTA, diethylenetriaminepentaacetic acid (DTPA), 1,4,7,10-tetraazacyclododecane-1,4,7,10-tetrakis(2-propionic acid) (DOTMA), 1,4,8,11-tetrazacyclotetradecane-1,4,8,11-tetraacetic acid (TETA), 1,4,7,-tricarboxymethyl 1,4,7,10 teraazacyclododecane triacetic acid (DO3A), 1,4,7,10-tetraazacyclo-dodecan-1-(2-hydroxypropyl)-4,7,10-triacetic acid (HP-DO3A), ethylenediamine-tetraacetic acid (EDTA), bis-2 (hydroxybenzyl)-ethylene-diaminediacetic acid (HBED), or 1,4,7-triazacyclo-nonane N,N′,N″-triacetic acid (NOTA).

In an embodiment, the chelating molecule may form a complex with a paramagnetic ion. A paramagnetic ion may be a metal ion which may magnetize parallel or antiparalell to a magnetic field. The paramagnetic ion may be a multivalent ion of paramagnetic metal. The paramagnetic metal may be selected from but is not limited to lanthanides and transition metals. The transition metals may include but are not limited to manganese, iron, chromium, nickel and cobalt. The lanthanides may include but are not limited to praseodymium, neodymium, samarium, gadolinium, terbium, dysprosium, holmium, erbium, europium and ytterbium.

In an embodiment, the contrast agent may be gadolinium, a highly paramagnetic ion. This embodiment may be utilized in an MRI procedure. Gadolinium may be combined with a chelating molecule. Gadolinium (Gd) may be combined with (2,2′,2″-(2-(2-(2-mercaptoethylamino)-2-oxoethyl)-1,4,7-tetraazacyclododecane-1,4,7-triyl)triacetic acid)(DOTA) and may form a Gd-DOTA complex. Gd-DOTA may form a stable contrast agent. Gd-DOTA may be used in humans.

A nanoconjugate herein having a high molecular weight and including a Gd-DOTA molecule may improve both the efficacy of BBB permeation and prolong the circulation time. This may improve the accumulation of the contrast agent inside brain tumor regions or in other areas with pathological conditions due to the high molecular weight of the nanoconjugate.

The one or more targeting modules attached to the polymalic acid-based molecular scaffold may include biological activities other than targeting. The one or more targeting modules may be configured to perform delivery of a pro-drug. The one or more targeting modules may include a releasable functional module that may become effective in the cytoplasm. The one or more targeting modules may be configured to direct the nanoconjugate towards a specific tissue by being capable of binding to the surfaces of cells. The one or more targeting modules may be configured to facilitate internalization of the nanoconjugate into the targeted cell through endosomes. The one or more targeting modules may be configured to promote escape of the nanoconjugate from endosomes into the cytoplasm by virtue of hydrophobic functional units that integrate into and disrupt endosomal membranes. The one or more targeting modules may be configured to increase effectiveness during acidification of endosomes en route to lysosomes. The one or more targeting modules may be configured to protect against degradative enzyme activities, for example, peptidases and proteases.

In an embodiment, a targeting module may be but is not limited to an antibody, a polypeptide, an oligonucleotide, a therapeutic chemical, or a phage. The one or more targeting modules may be capable of targeting a component of a diseased cell or a tissue.

In an embodiment, a targeting module may be an antibody. The antibody may have an ability to recognize and specifically bind to a target. The target may be but is not limited to a protein, a polypeptide, a peptide, a carbohydrate, a polynucleotide, a lipid, or combinations of at least two of the foregoing through at least one antigen recognition site within the variable region of the antibody.

In an embodiment, a targeting module may be an antibody of a class described as antagonist antibodies, which specifically bind to a cancer stem cell marker protein and interfere with, for example, ligand binding, receptor dimerization, expression of a cancer stem cell marker protein, and/or downstream signaling of a cancer stem cell marker protein.

In an embodiment, a targeting module may be an antibody of a class described as agonist antibodies which specifically bind to a cancer stem cell marker protein and promote, for example, ligand binding, receptor dimerization, and/or signaling by a cancer stem cell marker protein. In an embodiment, a targeting module may be an antibody that does not interfere with or promote the biological activity of a cancer stem cell marker protein and may instead function to inhibit tumor growth by, for example, antibody internalization and/or recognition by the immune system.

A targeting module may be selected from any type of antibody. The antibody may be a polyclonal antibody, an intact monoclonal antibody, an antibody fragment, which may be but is not limited to Fab, Fab′, F(ab′)2, an Fv fragment, a single chain Fv (scFv) mutant, a chimeric antibody or a multispecific antibody. A multispecific antibody may be a bispecific antibody generated from at least two intact antibodies. A targeting module may be a humanized antibody or a human antibody. A targeting module may be a fusion protein comprising an antigen determination portion of an antibody. A targeting module may be fragment of an antibody comprising an antigen recognition site. Antibodies selected from may include any of the five major classes of immunoglobulins: IgA, IgD, IgE, IgG, and IgM, or subclasses (isotypes) thereof (e.g. IgG1, IgG2, IgG3, IgG4, IgA1 and IgA2), based on the identity of their heavy-chain constant domains referred to as alpha, delta, epsilon, gamma, and mu. A targeting module may be a naked antibody or an antibody conjugated to other molecules. A targeting module may be an antibody conjugated to, for example, toxins or radioisotopes.

In an embodiment, a targeting module may be a monoclonal antibody. In an embodiment, a targeting module may be a polyclonal antibody. In an embodiment, a targeting module may be an antibody specific to at least one vasculature protein in a cell. The vasculature protein may be a transferrin receptor protein. The transferrin receptor protein as used herein refers to the receptor expressed on endothelium cell surfaces, and at elevated levels on certain tumors (Lee J H et al. 2001 Eur J Biochem 268:2004; Kovar M K et al., 2003 J Drug Targeting 10:23). A monoclonal antibody targeting module (TfR-mAb) may bind the transferrin receptor protein and thereby achieve transcytosis through endothelium associated with BBB. An embodiment includes Tfr-mAb attached to a Gd-containing nanoconjugate that may bind specifically to transferrin receptor residing at the endothelial surface on the luminal side of brain capillaries thus binding the nanoconjugate thereto. Once bound, the nanoconjugate may efficiently cross the BBB endothelium by transcytosis. A Tfr mAb-containing nanoconjugate may be of the size of 20-30 nm (molecular weight 100,000), which is known to be well above the renal exclusion limit.

A TfR mAb targeting module may be a humanized (hu-Tfr-mA) or a chimeric antibody. To study in vivo imaging in mouse and rat models of Alzheimer's disease (AD models), or TNBC metastasized to brain, hu-TfR mAb of the nanoconjugate could be replaced by mouse- or rat-TfR mAb. The nanoconjugate may contain other polypeptides used for similar purposes.

A targeting module may include a lectin or another ligand specific to the transferrin receptor. A targeting module may be a ligand to one of any number of cell surface receptors or antigens.

A targeting module may be a small drug molecule or a chromophore molecule, or a protein molecule, or a lectin that are covalently joined to polymalic acid in constructing the nanoconjugate.

A targeting module may be an antibody configured to specifically bind a protein selected from but not limited to EGFR, human epidermal growth factor (HER), laminin 411, insulin-like growth factor (IGF) and tumor necrosis factor-alpha (TNF-α). The antibody binding EGFR may be Cetuximab. The antibody binding EGFR may be Panitumumab. The antibody binding EGFR may be Zalutumumab. The antibody binding EGFR may be nimotuzumab. The antibody binding EGFR may Bematuzumab. The antibody may be any antibody binding, directed to or inhibiting EGFR. The antibody binding EGFR may be any known or commercially available antibody.

The antibody binding HER may be HER2/neu antibody. In an embodiment, the anti-HER2/neu antibody may be Trastuzumab Herceptin®. The anti-HER2/neu antibody may be Pertuzumab®. It is noted that the anti-HER2/neu antibody may be a monoclonal or polyclonal antibody. Further, the anti-HER2/neu antibody may be a humanized antibody or a chimeric antibody. The antibody binding HER may be any known or commercially available antibody. The antibody binding laminin 411 may bind either laminin β1 subunit, or laminin α4 subunit, or both.

A targeting module may be a peptide configured to specifically bind a protein selected from but not limited to EGFR, human epidermal growth factor (HER), laminin 411, insulin-like growth factor (IGF) and tumor necrosis factor-alpha (TNF-α). The peptide may be any known or commercially available peptide.

A targeting module may be an oligonucleotide. The oligonucleotide may be an antisense oligonucleotide inhibiting expression of a target nucleic acid molecule. The oligonucleotide may be one of the antisense oligonucleotides inhibiting expression of lamin 411 that were described in PCT Appl. PCT/US04/29956, filed Sep. 13, 2004; and U.S. application Ser. No. 10/570,747, filed Jan. 30, 2007, issued as U.S. Pat. No. 7,547,511, and Ser. No. 12/473,992, filed May 28, 2009, which are incorporated by reference as if fully set forth.

A targeting module may include an endosomal escape unit as described in PCT application PCT/US09/40252, filed Apr. 10, 2009, which is incorporated by reference as if fully set forth. An endosomal escape may be a carrier module attached to the polymalic acid-based scaffold that becomes active by acidification during maturation of the endosomal vesicles towards lysosomes. The carrier module may include a plurality of leucine residues in a polypeptide linked to the polymalic acid-based molecular scaffold by amide bonds. The carrier module may include a plurality of valine residues in a polypeptide linked to the polymalic acid-based molecular scaffold by amide bonds. The carrier module may include a leucine ethylester linked to the polymalic acid-based molecular scaffold by amide bonds. During acidification of the endosomes en route to lysosomes, these stretches of the carrier module may become charge-neutralized and hydrophobic, and capable of disrupting membranes. Other molecules that become charge neutralized at lysomal pH's may be used in place of leucine or valine residues, or a leucine ethylester in construction of the compositions containing polymalic acid and an endosomal escape unit module.

A targeting module may be a module for protection against resorption by the reticuloendothelial system (RES) and/or enzyme degradation. For example, the module for protection against resorption may be but is not limited to a polyethylene glycol (PEG) molecule. PEG may be used to increase the in vivo half-life of conjugated proteins, to prolong the circulation time, and enhance extravasation into targeted solid tumors (Arpicco S et al. 2002 Bioconjugate Chem 13:757; Maruyama K et al., 1997 FEBS Letters 413:1771, which is incorporated by reference as it fully set forth). Other molecules known to increase half-life of the nanoconjugate may be used in design of nanoconjugates herein.

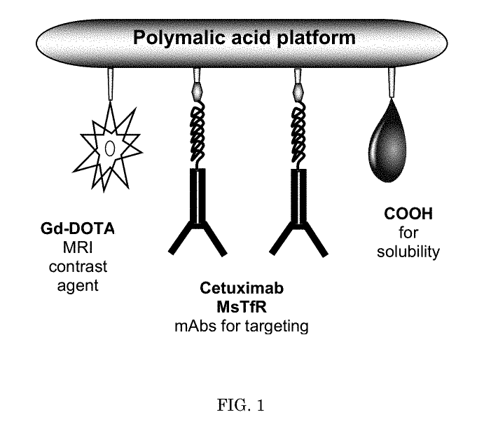

FIG. 1 depicts an exemplary nanoconjugate including Gd-DOTA complex attached to the polymalic acid platform. The nanoconjugate may be for tumor-type specific MRI in mouse model for human TNBC metastasized to brain. The modules attached to the polymalic acid may include an MRI contrast agent (Gd-DOTA), targeting modules (chimeric mouse-human monoclonal antibodies Cetuximab (Erbitux®) specific to EGFR displayed by tumor cells and MsTfR for penetration through BBB) and a carboxyl group for improving solubility. For use in humans, the anti-mouse TfR mAb may be replaced by anti-human TfR mAb.

Polymalic acid of any molecular weight (Mw) may be used as the platform to carry one or more targeting modules and one or more imaging moieties. Polymalic acid used herein may have a Mw of 10,000; 15,000; 20,000; 30,000; 40,000; 50,000; 60,000; 70,000; 80,000; 90,000; 100,000; 110,000; 120,000; 130,000; 140,000; or 150,000, or more, or any value in a range between any two of the foregoing (endpoints inclusive). The polymalic acid of Mw 80,000 may be platform for a nanoconjugate that caries covalently bound MsTfR mAb and a tumor specific mAb together with multiple covalently bound Gd-DOTA. The platform may contain any number of derivatisable carboxyl group. In embodiments, the platform may contain 700 or more derivatisable carboxyl groups and a large number of Gd-DOTA units can be loaded for generating a strong MRI signal.

In an embodiment, one or more targeting modules may be capable of targeting a component of a diseased cell or tissue. The component may be, but not limited to, beta amyloid plaques thought to contribute to the degradation of the neurons in the brain and the subsequent symptoms of Alzheimer's disease. The one one or more targeting modules may include curcumin (5-hydroxy-1,7-bis(4-hydroxy-3-methoxyphenyl)-1,4,6-heptatrien-3-on) for specific binding to Alzheimer's amyloid plaques. Curcumin may bind specifically and tightly to the beta amyloid plaques and thereby may allow accumulation of the nanoconjugate within the brain and a high staining intensity. The nanoconjugate may contain one or more curcumin molecules. The presence of multiple curcumin molecules on the nanoconjugate results in firm attachment of the nanoconjugates around to a beta-amyloid plaque contributing to sharp contours with high contrast.

The nanoconjugate molecule containing curcumin may carry any number of gadolinium ions. The nanoconjugate may carry a single gadolinium ion. The nanoconjugate may carry a plurality of gadolinium ions. The nanoconjugate may carry 1, 5, 10, 20, 30, 40, 50, 60, or more Gd ions per molecule of nanoconjugate. The nanoconjugate may carry a number of Gd ions per molecule of nanoconjugate in a range between any two of the following numbers: 1, 5, 10, 20, 30, 40, 50, or 60. A high concentration of Gd on a target tissue, for example amyloid plaques, may allow imaging by MRI at high contrast and resolution quality.

The one or more targeting module may include therapeutic polypeptides. In embodiments, the one or more targeting modules may include additional therapeutic agents. In embodiments, the additional therapeutic agent or agents is selected from the group consisting of growth factors, anti-inflammatory agents, vasopressor agents, collagenase inhibitors, topical steroids, matrix metalloproteinase inhibitors, ascorbates, angiotensin II, angiotensin III, calreticulin, tetracyclines, fibronectin, collagen, thrombospondin, transforming growth factors (TGF), keratinocyte growth factor (KGF), fibroblast growth factor (FGF), insulin-like growth factors (IGF), epidermal growth factor (EGF), platelet derived growth factor (PDGF), neu differentiation factor (NDF), hepatocyte growth factor (HGF), and hyaluronic acid.

In an embodiment, the nanoconjugate may include a tracking fluorescent dye to follow in vivo distribution of the nanoconjugate in a subject. The tracking dye may facilitate gross in vivo monitoring of the nanoconjugate distribution by imaging systems other than by using MRI. In the absence of Gd, the tracking dye may allow the validation of curcumin-polymalic acid conjugate entrance into the brain in the first phase of the investigation of a disease or condition in a subject. A tracking dye may also validate whether curcumin is attached to polymalic acid within the brain. Thus, the tracking dye may be useful in optimization experiments. Tracking may be performed, for example, by using Xenogen fluorescence imaging system.

In an embodiment, a kit for facilitating imaging of a cell or tissue is provided. The cell may be a diseased cell. The tissue may be a diseased tissue. The kit may be implemented in a method for visualizing pathological conditions. The kit may include a nanoconjugate comprising a polymalic acid-based molecular scaffold, one or more imaging moiety and one or more targeting module. The kit may include any one or more nanoconjugates described herein. At least one of the imaging moieties and at least one of the targeting modules may be conjugated to the polymalic acid-based molecular scaffold. All of the imaging moieties may be conjugated to the polymalic acid-based molecular scaffold. All of the targeting modules may be conjugated to the polymalic acid-based molecular scaffold.

The exact nature of the modules and moieties configured in the kit may depend on its intended purpose. In embodiments, the kit may be configured for the purpose of visualizing, treating or monitoring Alzheimer's disease or other conditions involving abnormal brain function, activity or pathology. For this purpose, the kit may include a nanoconjugate comprising a module for binding amyloid beta plaque and MRI imaging. In embodiments, the kit may be configured for the purpose of visualizing, treating, or monitoring cancer.

In an embodiment, the kit may be configured particularly for the purpose of treating mammalian subjects. The kit may be configured particularly for the purpose of treating human subjects. The kit may be configured for veterinary applications. The kit may be configured to, but is not limited to, treating farm animals, domestic animals, or laboratory animals. Instructions for use may be included in the kit. Instructions for use may include a tangible expression describing the technique to be employed in using the components of the kit to effect a desired outcome. For example, instructions may describe the technique to visualize amyloid beta plaques or tumor cells or cell types. The kit may also contain other useful components. For example, the kit may contain diluents, buffers, pharmaceutically acceptable carriers, syringes, catheters, applicators, pipetting or measuring tools, bandaging materials or other useful paraphernalia as will be readily recognized by those of skill in the art.

The materials or components assembled in the kit may be provided to the practitioner stored in any convenient and suitable ways that preserve their operability and utility. For example, the components may be provided be in dissolved, dehydrated, or lyophilized form. The components may be provided at room, refrigerated or frozen temperatures. The components may be contained in suitable packaging material(s). As used herein, the phrase “packaging material” refers to one or more physical structures used to house the contents of the kit, such as inventive compositions and the like. The packaging material may be constructed by well known methods, preferably to provide a sterile, contaminant-free environment. As used herein, the term “package” refers to a suitable solid matrix or material such as glass, plastic, paper, foil, and the like, capable of holding the individual kit components. The packaging material may have an external label which indicates the contents and/or purpose of the kit and/or its components.

In an embodiment, a composition comprising any one of the nanoconjugates described herein is provided. The composition may be combined with a pharmaceutically acceptable excipient. The pharmaceutically acceptable excipients may include NaCl, water, salt solutions, normal saline solutions, disintegrants, Ringer's solution, lactated Ringer's, sweeteners, normal sucrose, normal glucose, binders, fillers, lubricants, coatings, flavors, alcohols, oils, gelatins, carbohydrate, e.g., lactose, amylose or starch, fatty acid esters, hydroxymethycellulose, polyvinyl pyrrolidine, and colors. Other pharmaceutical excipients known in the art may be used.

The excipients may be sterilized, mixed with auxiliary agents, i.e., lubricants, preservatives, stabilizers, wetting agents, emulsifiers, salts for influencing osmotic pressure, buffers, coloring, and/or aromatic substances and the like that do not deleteriously react with the compositions described herein.

In an embodiment, the compositions described herein may be used in an intravenous dosage form.

In an embodiment, a method of targeting a cell or a tissue in a subject is provided. The cell may be a diseased cell. The tissue may be a diseased tissue. The method may involve administering to the subject a nanoconjugate that includes a polymalic acid-based molecular scaffold, at least one imaging moiety, and at least one targeting module. At least one of the imaging moieties and the at least one of the targeting modules may be conjugated to the polymalic acid-based molecular scaffold. All imaging moieties may be conjugated to the polymalic acid-based molecular scaffold. All targeting modules may be conjugated to the polymalic acid-based molecular scaffold. The nanoconjugate may be any nanoconjugate described herein. The method may also include providing conditions permitting interaction of the nanoconjugate with a component of the diseased cell or a diseased tissue.

The subject may be a patient. As used herein, the term “patient” refers to a human. The patient may be a human with a symptom or symptoms of a disease or condition. The patient may need treatment for the disease or condition in a clinical setting. The symptoms of the disease or condition may change as a result of a treatment, or spontaneous remission, or development of further symptoms with the progression of the disease. The term “patient” may also refer to non-human organism. The patient may be a laboratory animal, a farm animal or a zoo animal. The patient may be a mouse, a rat, a guinea pig, a hamster, a horse, a rabbit, a goat, or a cow.

In an embodiment of the method of targeting a cell or a tissue, a nanoconjugate may be administered to a subject by any suitable route. The nanoconjugated may be administered by intravenous injections. The nanoconjugate may be delivered by a technique selected from the group consisting of: intramuscular injection, subcutaneous injection, intravenous injection, intradermal injection, intranasal injection, inhalation, oral administration, sublingual administration, buccal administration, or topical administration.

In an embodiment of the method of targeting a cell or a tissue, the at least one imaging moiety may be a molecule facilitating an imaging technique. An imaging may be performed by any technique including but not limited to X-ray imaging, computer tomography (CT) scans, and MRI. The imaging moiety may include an imaging contrast agent. The imaging contrast agent may be a Gd-DOTA. The method may involve visualizing the imaging contrast agent in the subject. Visualizing may be performed by the imaging technique; e.g., by X-ray, CT, or MRI.

In an embodiment, the method of targeting a cell or a tissue may also include diagnosing a disease or other condition in the subject. Diagnosing may be based on an image of the diseased cell or the diseased tissue. Diagnosing may include comparing the image with a control image of a normal cell or tissue in a healthy individual. The image may be obtained by any non-invasive clinical diagnostic imaging procedure. For example, the image may be obtained by MRI. The MRI apparatus utilizes the nuclear magnetic resonance phenomenon and may produce images of cross sections of the cells or tissues being imaged. The MRI may measure signal derived from protons of the water molecules present in cells or tissues in a subject positioned for imaging. The intensity of MRI images may depend on physical properties of specific tissues. The intensity of MRI signal may depend on proton density, spin lattice relaxation time (T1), and the spin-spin relaxation time (T2).

An “abnormal condition” refers to a function in the cells and tissues in a body of a patient that deviates from the normal function in the body. An abnormal condition may refer to a disease. Abnormal condition may include brain disorders. Brain disorders may be but are not limited to Alzheimer's disease, Multiple sclerosis, Parkinson's disease, Huntington's disease, schizophrenia, anxiety, dementia, mental retardation, and anxiety. Abnormal condition may include proliferative disorders. The terms “proliferative disorder” and “proliferative disease” refer to disorders associated with abnormal cell proliferation. Proliferative disorders may be, but are not limited to, cancer, vasculogenesis, psoriasis, and fibrotic disorders. Cancer is a physiological condition in mammals in which a population of cells is characterized by unregulated cell growth. Examples of cancers include, but are not limited to, carcinoma, lymphoma, blastoma, sarcoma, and leukemia. More particular examples of such cancers include squamous cell cancer, small-cell lung cancer, non-small cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney cancer, liver cancer, prostate cancer, vulval cancer, thyroid cancer, hepatic carcinoma and various types of head and neck cancers. Breast cancer may include TNBC and HER2-positive breast cancer.

Cancer may be a primary cancer or a metastatic cancer. The term “primary cancer” refers to the original site at which a cancer originates. For example, a cancer originating in the breast is called a primary breast cancer. If it metastasizes; i.e., spreads to the brain, the cancer is referred to as a primary breast cancer metastatic to the brain.

The term “tumor” refers to any mass of tissue that result from excessive cell growth or proliferation, either benign (noncancerous) or malignant (cancerous) including pre-cancerous lesions. Tumor cell may derive from a tumor or a pre-cancerous lesion including both a non-tumorigenic cell and a tumorigenic cell; i.e., cancer stem cell.

Cancer may be a brain cancer. The brain cancer may be a primary brain tumor or a metastatic brain tumor. The primary brain tumor may be a glioblastoma. The metastatic brain tumor may be a triple negative breast cancer (TNBC) metastasized to the brain, HER2-positive breast cancer metastasized to the brain, or lung cancer metastasized to the brain.

The term “metastasis” refers to the process by which a cancer spreads or transfers from the site of origin to other regions of the body with the development of a similar cancerous lesion; i.e., having the same or substantially the same biochemical markers at the new location. A “metastatic” or “metastasizing” cell is one that has a reduced activity for adhesive contacts with neighboring cells and migrates by the bloodstream or within lymph from the primary site of disease to additional distal sites, for example, to invade neighboring body structures or distal structures.

An abnormal condition may also include diabetes, rheumatoid arthritis, asthma, psoriasis, atherosclerosis, cardiovascular disorders, glaucoma, and retinopathy. The term “disease” refers to all abnormal conditions. Diagnosing may include diagnosing of another condition in addition to an abnormal condition. The other condition may be associated with an abnormal condition. The other condition may not be associated with an abnormal condition. For example, diagnosing of schizophrenia may be made in addition to diagnosing Alzheimer's disease.

In an embodiment, a method of imaging a cell or a tissue in a brain of a subject and detecting cancer is provided. The method comprises co-administering to a subject a contrast agent and any one of the compositions described herein. The contrast agent may be any Gd-based non-specific contrast agent. The Gd-based contrast agent may be but is not limited to gadoterate (Dotarem®), gadodiamide (Omniscan™), gadobenate (MultiHance®), gadopentetate (Magnevist®), gadoteridol (ProHance®), gadoversetamide (OptiMARK®), or gadobutrol (Gadavist®).

As used herein, “co-administering” means that the composition described herein is administered at the same time, just prior to, or just after the administering of one or more additional contrast agents or therapeutic agents. The compositions of the invention may be administered alone or may be co-administered to the patient with additional agents or compounds. Co-administering is meant to include simultaneous or sequential administering of the compositions individually or in combination with other contrast agents or compositions having different targeting modules or therapeutic modules. The composition may also be combined, when desired, with other nanoconjugates, contrast agents, or active substances.

In an embodiment, co-administering includes administering a composition within 0.5, 1, 2, 4, 6, 8, 10, 12, 16, 20, or 24 hours of a contrast agent. Co-administering includes administering two compositions described herein simultaneously, approximately simultaneously (e.g., within about 1, 5, 10, 15, 20, 30, 60, 90, 120, 150, 180, 210, 240, 270, 300, 330, or 360 minutes of each other), or sequentially in any order. The step of co-administering may include sequential administering of the contrast agent and the composition. Sequential administration may 30, 40, 50, 60, 90, 120, 150, 180, 210, 240, 270, 300, 330 or 360 minutes, or any value between any two of the foregoing time points, subsequent to the administration of the contrast agent. Co-administering may be accomplished by co-formulation, i.e., preparing a single composition including nanonoconjugates carrying different targeting modules. In other embodiments, the compositions may be formulated separately. In an embodiment, one or more targeting modules, contrast agents or therapeutic agents may be linked or conjugated to one another.

In an embodiment, the method may also comprise visualizing the contrast agent and the imaging moiety of the any one the compositions described herein in the brain of the subject by an MRI technique. The method may comprise detecting the presence of a cancer in the brain of the subject. The step of detecting may comprise obtaining a first MRI image of the cell or the tissue in the brain after administering the contrast agent, and obtaining a second MRI image of the same location in the brain after administering the composition including the targeting module that targets cancer. The step of detecting may comprise measuring the intensity of an MRI signal in the first image and the second image and comparing the intensity and location of the MRI signal in the first image with the intensity and location of the MRI signal in the second MRI image. The intensity of the MRI signal in the first image may be measured at least 30 minutes subsequent to the administration of the contrast agent. The intensity of the MRI signal in the first image may be measured 20, 40, 60, 80, 100, 120, 140, 160, 180, 200, 220, 240, 260, 280, 300, 320, 340, or 360 minutes, or any value between any two of the foregoing time points, subsequent to the administration of the contrast agent. The intensity of the MRI signal in the second or subsequent images may be measured at a time of 20, 40, 60, 80, 100, 120, 140, 160, 180, 200, 220, 240, 260, 280, 300, 320, 340, or 360 minutes, or any value between any two of the foregoing time points, subsequent to the administration of the composition comprising the targeting module. The similarity in the intensity and location of the MRI signal in the first MRI image relative to the second MRI image may be indicative of the presence of the cancer in the brain of the subject.

In an embodiment, the method may comprise administering a composition comprising the targeting module that is an antibody that specifically binds to a protein selected from the group consisting of: an epidermal growth factor receptor, laminin 411, insulin-like growth factor, transferrin receptor protein, and tumor necrosis factor-alpha. The method may comprise administering the composition comprising the antibody that is Cetuximab or Trastuzumab, or both. The composition may comprise at least two targeting modules, and may include an antibody that specifically binds the transferrin receptor protein.

In an embodiment, the method may comprise administering the MRI contrast agent, for example, gadobenate (MultiHance®) to the subject. The method may include obtaining a first MRI image of the brain. The method may further comprise measuring the intensity of the MRI signal in the first image. The method may comprise analyzing the intensity of the MRI signal in the first MRI image and comparing it with control images. The step of analyzing may comprise calculating signal to noise ratios (SNRs) and contrast-to-noise ratios (CNRs) for regions of interest (ROIs) in the brains of the subject. The ROI may be a tumor. The SNR may be assessed as SI_{ROI}/SD, wherein SI_{ROI} is the mean signal intensity in the brain ROI and the SD is the standard deviation of the signal intensity within the background (air). The CNR may be assessed as SI_{ROI}−SI_{control}/SD, wherein the SI_{ROI} is the mean signal intensity for ROI, e.g., brain tumor, and SI_{control} is signal intensity for the neighboring region of the brain. The method may comprise subtracting signal to noise ratios (SNRs) obtained for the pre-contrast MRI from the post MRI SNRs obtained for regions of interest (ROI) in the brain of the patient. Similarly, pre-contrast CNRs may be subtracted from post-contrast CNRs. A tumor body may be identified upon analysis of the first MRI image and finding the statistically significant differences in SNR and CNR values for the contrast agent.

In an embodiment, the method may include administering the composition comprising the targeting module that binds to a specific tumor variant. The method may include administering the composition comprising Trastuzumab that binds specifically to the HER2-expressing tumor. The composition may be administered 60, 90, 120, 150, or 180 minutes subsequent to the injection of the MultiHance® contrast agent. The method may include obtaining a second MRI image of the brain. The method may further comprise measuring the intensity of the MRI signal in the second image. The method may comprise analyzing the intensity of the MRI signal in the second MRI image similarly to the analysis performed for the first MRI image, and comparing the intensity and location of the MRI signal in the second image with the intensity and location of the MRI signal in the first MRI image. The similarity in the intensity and location of the MRI signal in the second MRI image relative to the first MRI image may be indicative of the presence of the HER2-expressing tumor in the brain of the subject.

In an embodiment, the method may comprise administering the composition comprising the targeting module that is Cetuximab that binds specifically to the EGFR-expressing tumors. The method may include obtaining the second MRI image, analyzing the image, measuring the intensity of the signal in the second MRI image as described herein and comparing the intensity and location of the MRI signal to that of the MRI signal obtained for the first MRI image. The similarity in the intensity and location of the MRI signal in the first MRI image relative to the second MRI image may be indicative of an EGFR-expressing tumor. Since the EGFR-expressing tumor may be a primary cancer, such as glioblastoma, or a metastatic brain cancer, such as a triple negative breast cancer metastasized to the brain, or lung cancer metastasized to the brain, the method may further comprise differentially diagnosing the primary brain cancer from a metastatic brain cancer. The differential diagnosing may include administering to the subject a composition comprising a targeting module that is an antibody that specifically binds to laminin 411. The composition may be administered 60, 90, 120, 150, or 180 minutes subsequent to the injection of the composition comprising Cetuximab. The method may include obtaining a third MRI image, measuring the intensity of an MRI signal in the the third image; and comparing the intensity and location of the MRI signal in the second image with the intensity and location of the MRI signal in the prior first and second MRI images. The similarity in the intensity and location of the MRI signal in the second MRI image relative to the third MRI image may be indicative of the presence of the glioblastoma in the brain of the subject.

In an embodiment, the method may include composition comprising any targeting modules. The composition may include a targeting module that is an anti-insulin antibody recognizing a brain tumor overexpressing insulin receptor. Still another composition may include a targeting module that targets tumor necrosis factor may recognize inflammation when compared to the first MRI signal generated by the non-specific contrast agent.

An embodiment includes a tumor-specific nanoconjugate, which may be implemented for enhancement of MRI and facilitating diagnostic imaging. An enhancement includes such a method. In particular, a tumor-specific nanoconjugate may be used to distinguish between tumor and non-tumor lesions of the brain which are indistinguishable by a common MRI procedure. A nanoconjugate may be used to distinguish between different types of tumors occurring side-by side in the same individual. A nanoconjugate may be used for MRI enhancement in the brain of cancer patient with a history of primary breast cancer, metastatic brain tumor from primary breast cancer, metastatic tumors from a different type of cancer, a primary brain tumor, and/or infection resulting from impairment of the immune system as a complication of chemotherapy.

A nanoconjugate herein may be designed to enhance MRI-based diagnostics of specific conditions. In an embodiment, a nanoconjugate (MRI enhancer) may include antibodies specific for tumor markers at the surface of tumor cells. The antibodies may be specific to overexpressed cell-surface antigens such as EGFR, HER2, B lymphocyte antigen CD 20 or laminin. The antibodies may facilitate access to the tumor tissue across the BBB into tumor interstitial using transcytosis through targeting of transferrin receptor on the endothelium of tumor capillaries. Once attached, the enhancer could be retained over a time scale that exceeds by far the clearance of unbound free MRI enhancer through the kidneys. On basis of the prolonged retention in the brain or other tumors, MRI could recognize the labeled tumor by a signal sent as a shortened relaxation time T1 of the reagent surrounding water molecules after given pulses of a spin orientating external magnetic field of the MRI apparatus. The shortening of the reciprocal of T1 is proportional to the concentration of the MRI enhancer, and thus the enhancement of the signal may be the result of an accumulation due to tumor specific binding. The tumor nonspecific MRI signal may be accounted for by measurement of T1 measured for healthy portions of the brain. The difference of T1 values between tumor and healthy brain may be measured as a function of time reflecting specific tumor retention of the enhancer reagent, while the reagent in the healthy brain and elsewhere may be already cleared through the kidneys. Tumor-type specific MRI scanning may be performed when T1 for the healthy brain has approached zero value.

A number of contrast agents may be included in a nanoconjugate herein to improve resolution of MR images. A contrast agent may be a molecule suitable to generate a contrasting effect in vivo. A contrast agent may form metalloprotein complex. A contrast agent may form a complex that affects the relaxation times T1, or T2, or both. A contrast agent that affects T1 may be a lanthanide metal ion. A contrast agent may be Gd that is chelated to a low molecular-weight molecule in order to limit toxicity. A contrast agent that affects T2 may consist of small particles of magnetite (FeO—Fe₂O₃). Contrast agents may interact with mobile water in tissue to produce contrast.

In an embodiment, diagnosing the disease or condition may involve a patient with abnormal brain function, activity or pathology. Diagnosing the Alzheimer disease may be based on the presence of amyloid beta plaques in the patient's brain.

Diagnosing may be performed by administering a composition that includes a polymalic-acid based nanoconjugate containing a targeting module for binding amyloid beta plaques and an imaging moiety for MRI imaging to the patient and acquiring images of localization of the nanoconjugate in a particular type of tissue in the patient's body.

The nanoconjugate may be able to pass the BBB and then target plaques, a hallmark of Alzheimer's disease, by simultaneously having attached plaque-binding curcumin and TfR mAb. Access to beta-amyloid plaque imaging may allow determining the status of Alzheimer's disease and to follow patients during the treating the disease. Similar Polycefin nanoconjugates containing curcumin and/or other active compounds could be used to treat Alzheimer's disease.

In an embodiment, application of a nanoconjugate may improve both the efficacy of BBB permeation and may prolong circulation of the Gd-containing contrast agent. It may also improve the accumulation inside brain regions that contain plaques due to tight binding to Alzheimer's amyloid plaques to curcumin.

In an embodiment, targeting the diseased cell or tissue may result in reduction or elimination of at least one symptom of the disease or condition, and thereby treatment of the disease or condition in the subject. Targeting the diseased cell or tissue may be a therapeutic measure to promote regression of a cancer or prevent further development or metastasis, or as a prophylactic measure to minimize complications associated with development of a tumor or cancer.

In an embodiment, the condition and/or disease monitored or treated may be Alzheimer's disease. In an embodiment, a method of treating a condition in a patient is provided. The method may include administering a composition comprising a nanoconjugate comprising a targeting module for binding amyloid beta plaques and an imaging moiety for MRI imaging. The method may also include treating the patient with the composition.

To achieve the desired effect; i.e., inhibit the expression of at least one ligand of the target receptor, a composition may be administered at a therapeutically effective amount. A “therapeutically effective amount” of the composition may be the amount effective for preventing further development of a cancer or transformed growth, and even to effect regression of the cancer.

The exact dosage may be chosen by the individual physician with regard to the need of the patient to be treated. Dosage and administration may be adjusted to provide sufficient levels of the active agent(s) or to maintain the desired effect. Additional factors which may be taken into account include the severity of the disease state; e.g., cancer size and location; age, weight and gender of the patient; diet, time and frequency of administration; drug combinations; reaction sensitivities; and tolerance/response to therapy. Long acting compositions might be administered every 3 to 4 days, every week, or once every two weeks depending on half-life and clearance rate of the particular composition.

In an embodiment, the one or more targeting modules may include active agents for treating a disease or condition in a patient. The active agents may be formulated in dosage unit form for ease of administration and uniformity of dosage. The expression “dosage unit form” as used herein refers to a physically discrete unit of active agent appropriate for the patient to be treated.

For any active agent, the therapeutically effective dose may be estimated initially either in cell culture assays or in animal models, usually mice, rabbits, dogs, or pigs as shown in Examples herein. The animal model may be also used to achieve a desirable concentration range and route of administration. Such information may then be used to determine useful doses and routes for administration in humans. A therapeutically effective dose refers to that amount of active agent which ameliorates the symptoms or condition. Therapeutic efficacy and toxicity of active agents can be determined by standard pharmaceutical procedures in cell cultures or experimental animals, e.g., ED50 (the dose is therapeutically effective in 50% of the population) and LD50 (the dose is lethal to 50% of the population). The dose ratio of toxic to therapeutic effects is the therapeutic index, and it can be expressed as the ratio, LD50/ED50. Compositions herein may exhibit large therapeutic indices. The data obtained from the animal studies may be used in formulating a range of dosage for human use.

As discussed above and described in greater detail in the Examples, a nanoconjugate herein may be administered in a method to prevent development or metastasis of a cancer condition. In particular, a nanoconjugate may be useful in preventing further growth of a particular cancer type, including but not limited to breast cancer; skin cancer; ovarian cancer; cervical cancer; retinoblastoma; colon cancer and other conditions including those arising from the lining of the gastrointestinal tract; lung cancer and cancers of the respiratory tract; renal carcinoma and other tumors arising from the inner surface of kidney tubules; leukemias and lymphomas and such disorder of blood; and other types of genital cancer including those associated with various strains of papilloma virus; brain tumors; and cancers of the uterus, of the vagina, and of the urethra.

In embodiments, diagnostic, prognostic and therapeutic methods may not be limited to treating conditions in humans, but may involve similar conditions in any mammal including but not limited to bovine, canine, feline, caprine, ovine, porcine, murine, and equine species.

In an embodiment, a method of monitoring an efficiency of treatment of a disease or condition in a subject is provided. Monitoring may include obtaining a first image of a diseased cell or a diseased tissue in the subject after treatment, and, after a period of time, a second image of the diseased cell or tissue. Comparison can be made between the first and the second images to determine a clinically significant difference in cells and tissues after the treatment. For example, two or more images may be compared to determine whether the treatment reduced the number of cancer cells in a tumor, or the size of a particular tumor.

A subject may be a patient in need of MRI procedure. A composition that includes a polymalic acid-based molecular scaffold, at least one imaging moiety, and at least one targeting module may be administered at any time before or after placing a patient in an MRI apparatus. The composition may target cells or tissues at different locations of the patient's body before images may be produced. In this case, the composition may be accumulated in the specific location before imaging. The images may be also produced during the period of accumulation of the composition in target cells or tissues. Any disease cells or tissues targeted by the composition may be identified by examining the image or images. The composition may be re-administered to the subject after a period of time depending on the scheme of a particular therapeutic treatment. For example, the composition may be administered every week, every two weeks, every three weeks, or every month. Image(s) may be produced during or after subsequent administration of the composition and comparison may be made between images taken during different phases of therapeutic treatment to assess the efficacy of treatment.

Methods herein may include providing a period of time sufficient for accumulation of a nanoconjugate in targeted cells or tissues.

In another embodiment, a method of prognosing a condition and/or disease is provided for an individual having abnormal brain function, activity or pathology. The method may include administering a composition comprising a nanoconjugate comprising a targeting module for binding amyloid beta plaques and a module for MRI imaging to the individual, and prognosing a severe form of the condition and/or disease based on the presence of an extensive level of amyloid beta plaques in the individual relative to a normal subject.

In an embodiment, a composition including a polymalic acid-based molecular scaffold, at least one imaging moiety, and at least one targeting module may further include a pharmaceutically acceptable carrier. As used herein, the term “pharmaceutically acceptable carrier” includes any and all solvents, diluents, or other liquid vehicle, dispersion or suspension aids, surface active agents, isotonic agents, thickening or emulsifying agents, preservatives, solid binders, and lubricants as suited to the particular dosage form desired. A pharmaceutically acceptable carrier may be one described in Remington's Pharmaceutical Sciences Ed. by Gennaro, Mack Publishing, Easton, Pa., 1995, which is incorporated herein by reference as it fully set forth, and discloses various carriers used in formulating pharmaceutical compositions and known techniques for the preparation thereof. Some examples of materials which can serve as pharmaceutically acceptable carriers include but are not limited to sugars, lactose, glucose, and sucrose; starches, corn starch and potato starch; cellulose and its derivatives, sodium carboxymethyl cellulose, ethyl cellulose, and cellulose acetate; powdered tragacanth; malt; gelatin; talc; excipients, cocoa butter and suppository waxes; oils, peanut oil, cottonseed oil, safflower oil, sesame oil, olive oil, corn oil, and soybean oil; glycols, propylene glycol; esters, ethyl oleate and ethyl laurate; agar; buffering agents, magnesium hydroxide, and aluminum hydroxide; alginic acid; pyrogen-free water; isotonic saline; Ringer's solution; ethyl alcohol, and phosphate buffer solutions, as well as other non-toxic compatible lubricants, sodium lauryl sulfate and magnesium stearate. Coloring agents, releasing agents, coating agents, sweetening, flavoring and perfuming agents, preservatives and antioxidants may also be present in the composition.

In an embodiment, a method of synthesizing a nanoconjugate is provided. The method may include providing a polymalic acid having a plurality of pendant carboxyl groups. The method may include reacting a compound containing sulfhydryl groups and amino groups through the pendant carboxyl group to add sulfhydryl groups to the polymalic acid to form an activated polymalic acid. The method may also include reacting at least one imaging moiety containing a sulfhydryl binding group to the activated polymalic acid to form a preconjugate. The method may further include reacting at least one targeting module containing a sulfhydryl binding group to the activated polymalic acid.

The method of synthesizing may include activating pendant carboxyl carboxyl groups on polymalic acid by adding N-hydroxysuccinimide (NHS) to the polymalic acid to form an NHS-ester. The method may also include reacting the activated carboxyl groups with the amino group of 2-mercapto-ethane-1-amine. The method may also include reacting at least one imaging moiety containing an amino group with the NHS-activated pendant carboxyl group. The method also may involve reacting at least one targeting module containing a sulfhydryl group to the preconjugate. The at least one imaging moiety may include an activated molecule of a contrast agent. The activated molecule of the contrast agent may include gadolinium (Gd)-1,4,7,10-tetraazocyclododecane-1,4,7,10-tetraacetic acid (DOTA)-amine. The at least one targeting module may include an activated antibody. The activated antibody may include an antibody-polyethylene glycol-maleimide. The antibody-polyethylene glycol-maleimide may further react with the preconjugate to form the nanoconjugate.

The at least one targeting module may include an activated curcumin-polyethylene-glycol amine. The at least one targeting module may specifically bind to a component of a diseased cell or tissue in a subject selected from the group consisting of: an epidermal growth factor receptor (EGFR), human receptor growth factor (HER), laminin 411, insulin-like growth factor (IGF), transferrin receptor protein, curcumin and tumor necrosis factor-alpha (TNF-α).

A polymalic acid having one or more targeting modules may be synthesized by any known method. For example, a polymalic having attached one or more target modules may be synthesized by ring-opening polymerization of derivatized malic acid lactones. Doxorubicin-poly-malic acid may be synthesized from synthetic poly-β-D, L-malic acid.

Further embodiments herein may be formed by supplementing an embodiment with one or more element from any one or more other embodiment herein, and/or substituting one or more element from one embodiment with one or more element from one or more other embodiment herein.

### EXAMPLES

The following non-limiting examples are provided to illustrate particular embodiments. The embodiments throughout may be supplemented with one or more detail from one or more example below, and/or one or more element from an embodiment may be substituted with one or more detail from one or more example below.

### Example 1. Chemical Synthesis of a Tissue Specific Nanoconjugate for MRI Enhancement

Materials. High purity polymalic acid (PMLA; Mw 800,000, polydispersity factor P=1.2 by Sec-HPLC) isolated from the culture medium of Physarum polycephalum was used as Polycefin platform (Ljubimova J Y et al. 2007 Chem-Biol Interactions 171:195). Mouse anti-human TfR mAb RVS10 was purchased from Southern Biotech (Birmingham, Ala., USA) and ERBITUX® (Cetuximab) from Bristol-Myers Squibb (New York, N.Y., USA). mPEG₅₀₀₀-Amine and maleimide-PEG₃₄₀₀-maleimide were obtained from Laysan Bio, Inc. (Arab, Ala., USA). 3-(2-Pyridyldithio)-propionate (PDP; Carlsson J et al. 1978 Biochem J 173:723. Alexa Fluor® 680 C2 maleimide (Alex680) was purchased from Invitrogen Corporation (Carlsbad, Calif., USA), 2-Aminoethyl-mono-amide-DOTA-tris(t-Bu ester) from (Macrocyclics, Inc. TX, USA). Unless indicated, chemicals and solvents of highest purity were obtained from Sigma-Aldrich (St. Louis, Mo.) USA.

Analytical Methods for Chemical Synthesis.

The conjugation reaction of Gd-DOTA-amine and 2-MEA with PMLA was followed by thin layer chromatography (TLC) on precoated silica gel 60 F254 aluminum sheets (Merck, Darmstadt, Germany) under UV light and/or by ninhydrin staining. Size exclusion chromatography was performed on an Elite LaChrom analytical system with Diode Array Detector L 2455 (Hitachi, Pleasanton, Calif., USA), and M_{w} was measured using either BioSep-SEC-S 3000 or PolySep-GFC P4000 (300×7.80 mm; Phenomenex, Torrance, Calif., USA) using 50 mM sodium phosphate buffer pH 6.8 as mobile phase (0.75 mL/min) and polystyrene sulfonates as molecular weight standards. Thiol residues were assayed by the method of Ellman (Ellman G L 1959 Arch Biochem Biophys 82:70). TfR binding activity of anti-mouse TfR mAb conjugated to polymalic acid was assayed using Protein Detector™ ELISA Kit (KPL, Inc., Gaithersburg, Mass., USA). The mouse TfR ectodomain used as the antigen was obtained from Protein Expression Center, California Institute of Technology (Pasadena, Calif. USA). Binding of polymalic acid conjugated Cetuximab to EGFR expressed on triple-negative breast cancer cells was demonstrated by fluorescence activated cell sorting (FACS) analysis. Gadolinium was measured by ICP-MS at UCLA, Los Angeles, Calif., USA). In the absence of protein, the reaction of DOTA-Gd was followed by its intrinsic fluorescence at 280 nm excitation wavelength and 316 nm emission wave length (Hagan J J et al. 1988 Anal Biochem. 60:514).

### Example 2. Synthesis—an Overview

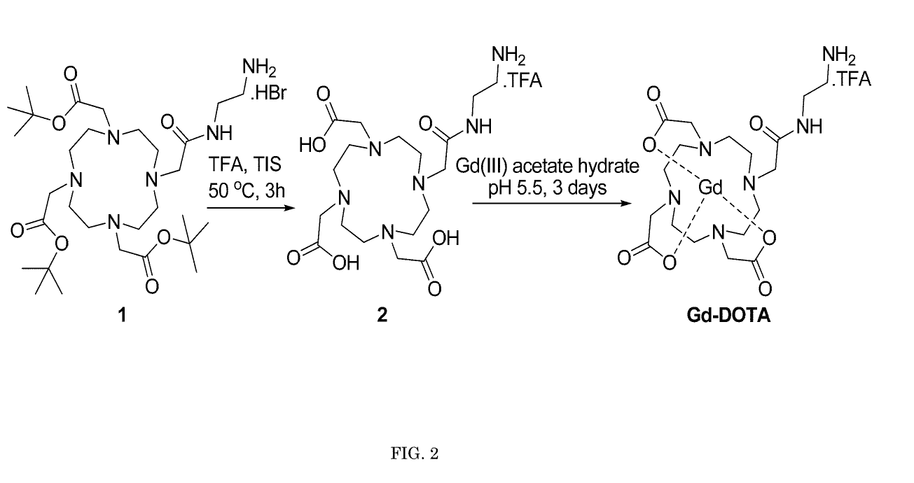

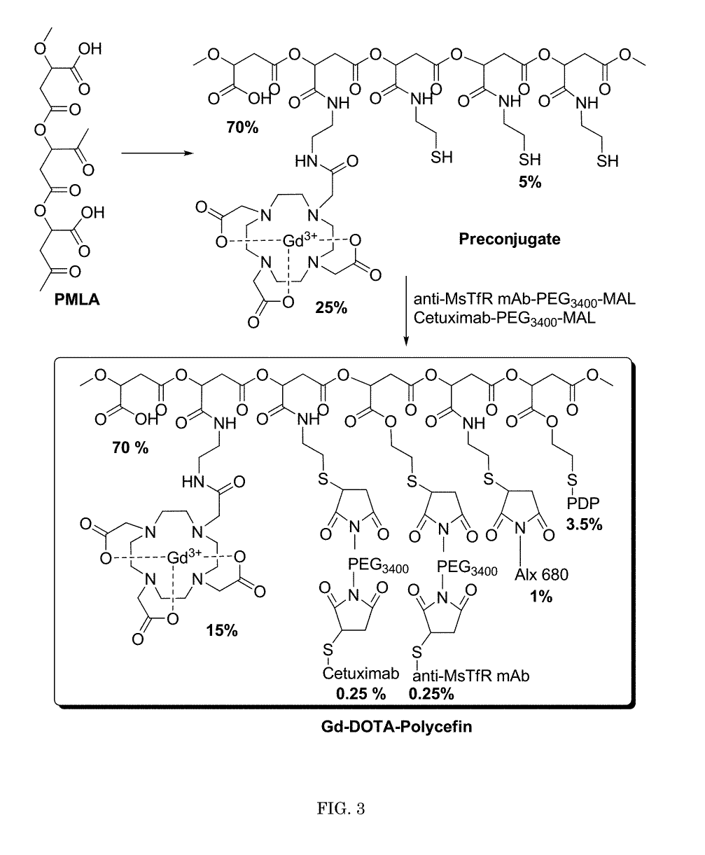

Synthesis of the tumor-type specific MRI enhancer reagent was accomplished in two parts: first the synthesis of Gd-DOTA-amine (FIG. 2) and second the conjugation of Gd-DOTA-amine to NHS-activated PMLA (FIG. 3). The alternative route includes first conjugating DOTA-amine with PMLA and then loading with Gd3+. The first part of the synthesis started with deprotection of the commercially available DOTA amino derivative (FIG. 2). The conjugation of Gd-DOTA amino with activated polymalic acid shown in FIG. 3 may be subject to variation for further increase in number of Gadolinium per polymer chain and for increase in reaction yields.

### Example 3. General Procedure for Boc Deprotection

Referring to FIG. 2, (1) 2-Aminoethyl-mono-amide-DOTA-tris(t-Bu ester) (1.23 g, 1.77 mmol) was dissolved in trifluoroacetic acid (TFA) (25 mL) and Triisopropylsilane (TIS) (1.12 g, 7.1 mmol) was added. The reaction mixture was stirred at 50° C. for 3 hours and cooled to room temperature. Evaporation of the solvent under reduced pressure yielded viscous brown oil. An ice-cold diethyl ether (25 mL) was added and the white precipitate was filtered and washed with diethyl ether. The dried precipitate was dissolved in pure water and freeze dried. Reaction yield was 97%.

### Example 4. General Procedure for Preparation of Metal Complex

Referring to FIG. 2, an equivalent of DOTA amine (2) (295 mg, 0.56 mmol) dissolved in 4 mL of water, received dropwise a slight stoichiometric excess of a Gadolinium (III) acetate (250 mg, 0.61 mmol) in 4 mL of water. The solution was stirred at room temperature (RT) while the pH was continuously maintained at pH 5.5 by adding 1M KOH. After stirring for 48 hours, EDTA (0.2 equivalent) was added and the mixture stirred at room temperature for 1 hour and then freeze dried. Reaction yield was 95%.

### Example 5. Synthesis of Preconjugate [P/Gd-DOTA (15%)/MEA (5%)]

N-Hydroxysuccinimide (NHS) (0.62 mmol) and N,N′-dicyclohexylcarbodiimide (DCC; 1 mmol) dissolved in 2 mL of dimethylformamide (DMF) were added consecutively to 72 mg of PMLA (0.62 mmol with regard to malyl units) in 1.5 mL of anhydrous acetone. After stirring at RT for 3 hours to complete the activation the mixture was filtered and acetone removed by rotary evaporation. A solution of Gd-DOTA in DMF 15 Mol-% (with regard to malyl units) was added dropwise at RT under stirring followed by addition of 0.15 mmol of triethylamine (TEA). The reaction was complete after 2 h according to TLC (ninhydrin, Rf=0 for the polymer conjugate, Rf=0.2 for Gd-DOTA; n-butanol:acetic acid:water 1:1:1). After addition of 2-mercapto-ethane-1-amine (MEA) 0.5 mmol of in DMF (100 μL, 5 Mol-% with regard to malyl units) and stirring at RT for 1 hour, buffer (100 mM sodium phosphate and 150 NaCl, pH 6.8) was added and stirring continued at RT for 30 min. After centrifugation at 1500×g for 10 minutes the clear supernatant was passed over a Sephadex column (PD-10, GE Healthcare, Piscataway, N.J., USA) equilibrated with deionized (DI) water. The product containing fractions containing the conjugate polymalic acid (P), Gd-DOTA (15%) and 2-mercapto-ethane-1-amine (MEA; 5%) were lyophilized (white powder). Reaction yield was 34.4%.

Referring to FIG. 3, the PMLA based preconjugate contains 25% of Gd-DOTA, 70% of derivatisable carboxyl groups and 5% of sulfhydryl groups.

### Example 6. General Procedure for Synthesis of Antibody-PEG₃₄₀₀-Maleimide

Referring to FIG. 3, antibodies (each of anti-MsTfR mAb and Cetuximab; 5 mg˜33 nmol, Mr˜150 kD) were dissolved in 2 mL of 100 mM sodium phosphate buffer containing 150 mM NaCl pH 5.5. Tris(2-carboxy ethyl) phosphine hydrochloride (TCEP, 50 mM in water) was added at a final concentration of 5 mM. After 30 minutes at room temperature. TCEP was removed over Sephadex PD10 and the reduced antibody was immediately added dropwise to maleimide (MAL)-PEG₃₄₀₀-MAL (10 mmol) dissolved in 5 mL sterile sodium phosphate buffer, 100 mM, 150 mM NaCl (pH 5.5) (always freshly prepared before use). After overnight stirring at 4° C., the mixture was concentrated over centrifuge membrane filter (Vivascience, cut off 30 kD, 20 mL, 100 mM sodium phosphate buffer containing 150 mM NaCl, ˜pH 5.5) and purified over Sephadex G75 equilibrated with 100 mM sodium phosphate buffer, 150 mM NaCl, pH 6.2. Reaction yield was 75-85%

### Example 7. General Procedure for Synthesis a Gd-DOTA-Polycefin Nanoconjugate

A total of 6 mg (2 mg/mL) of anti-mouse transferrin receptor mAb (anti-MsTfR mAb) and Cetuximab (each conjugated with PEG₃₅₀₀/maleimide) in 100 mM sodium phosphate buffer/150 mM NaCl (pH 6.2) was added to 10 mg (2-3 mg/mL) of a preconjugate P/Gd-DOTA (15%)/MEA (5%) in the same buffer. After 1 hour at room temperature, the extend of the reaction was analysed by SEC-HPLC. Alexa Fluor® 680 C2-maleimide (Alx 680) 1 mg in ml DMF was added and stirred for 1 h at RT. Remaining —SH-groups were blocked by adding excess of pyridyl(dithio)propionate (PDP) for 30 min at room temperature. After concentration over a centrifuge membrane filter Vivaspin 20, cutoff 30 kDa, 20 mL at 1500×g (Sartorius Stedim Biotech, Concord, Calif., USA), the final volume was adjusted to 2 ml before purification over Sephadex G-75 equilibrated with PBS, pH 7.4. Product containing fractions were isolated, combined and concentrated via membrane filtration. Reaction yield was 80-90%. FIG. 3 illustrates a synthesised Gd-DOTA-Polycefin nanoconjugate containing 15% Gd-DOTA, 0.25% Cetuxumab, 0.25% anti-MsTfR mAb, 1% Alexa Fluor® 680 (Alx 680), 3.5% PDP and 70% pendant carboxyl groups. Results of Gd-analysis indicated 12% loading with regard to polymalic acid carboxyls. 12% loading corresponds to an average of 82 molecules of Gd loaded on each enhancer molecule.

### Example 8. Characterization of Gd-DOT-Polycefin with Covalently Bound Cetuximab

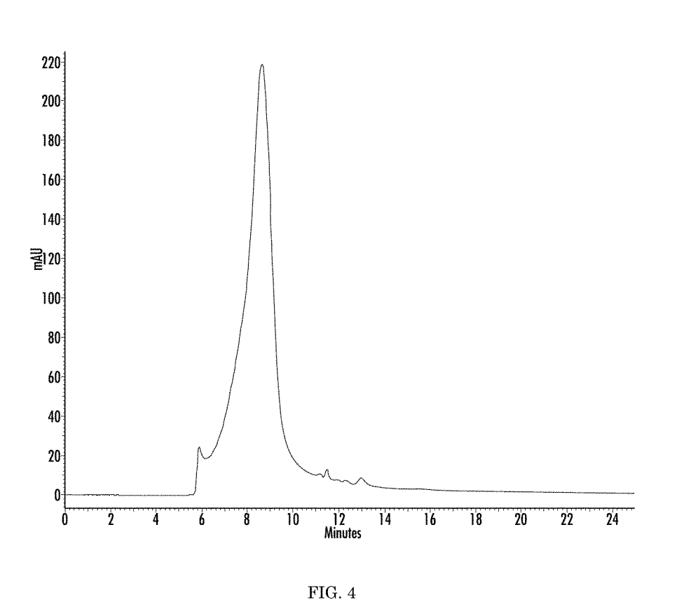

Purity of the synthesized nanoconjugate was assessed by HPLC profiling. FIG. 4 depicts the elution profile of Gd-DOTA-Polycefin molecule carrying covalently bound Cetuximab. The detection was performed at 220 nm wavelength. Referring to FIG. 4, the position of the peak eluted as an early fraction (8 min) indicates a high purity and high molecular weight (Mw 470,000) of the nanoconjugate.

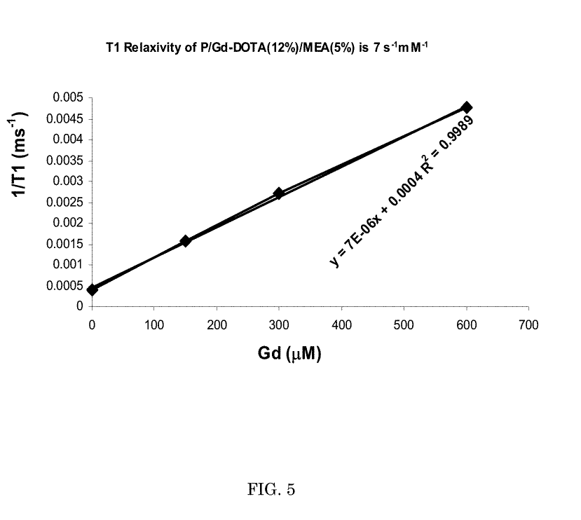

FIG. 5 shows calculation of T1-relaxivity of Polycefin-Gd-DOTA (12%)-MEA (5%). Relaxivity refers to a measure of the ability of magnetic compounds to increase the relaxation rates of the surrounding water proton spins in nuclear magnetic resonance applications. Referring to FIG. 5, the T1 relaxivity value was calculated to be equal to 7 s⁻¹ mM⁻¹. The calculated value is smaller than that of clinical MRI systems using a static magnetic field strength of 1.4 Tesla. A static magnetic field strength of the Siemens Microscan used was 9.4 Tesla. Relaxivity was calculated by measuring the slope of 1/T1 versus Gd concentration (μM). The equation Y=7E−0.6x+0.0004 allowed to translate absorbance at OD 450 directly to μM concentrations. The R² value equal to 0.9989 shows high accuracy of the calculation (with R² equal to 1 being perfect).

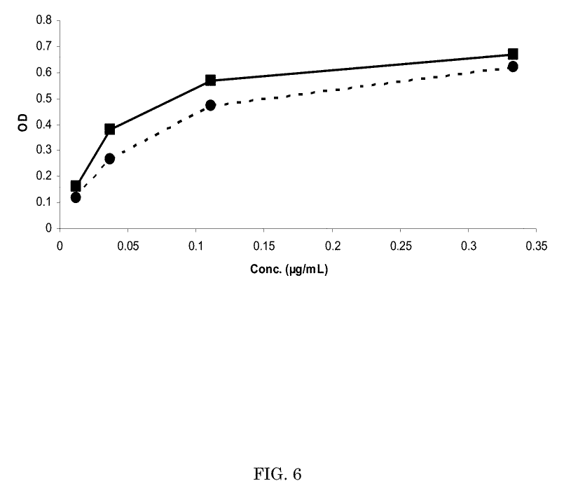

Affinity of ani-mouse TfR mAb to a target antigen (mouse-TfR) was determined by saturation ELISA (FIG. 6). The data shows that binding of a Gd-DOTA-Polycefin nanoconjugate containing Cetuximab, MsTfR and Alexa Fluor 680 was comparable to that o free anti-mouse TfR mAb. Referring to FIG. 6, it was observed that the values of the dissociation constants of the antigen-antibody complexes were similar and in the range of 0.03 to 0.08 μg/mL corresponding to 0.2 nM to 0.5 nM. These values are close to published values and indicate that the antigen binding of anti-Mouse TfR mAb was not affected by its attachment to the Gd-DOTA-Polycefin nanoconjugate.

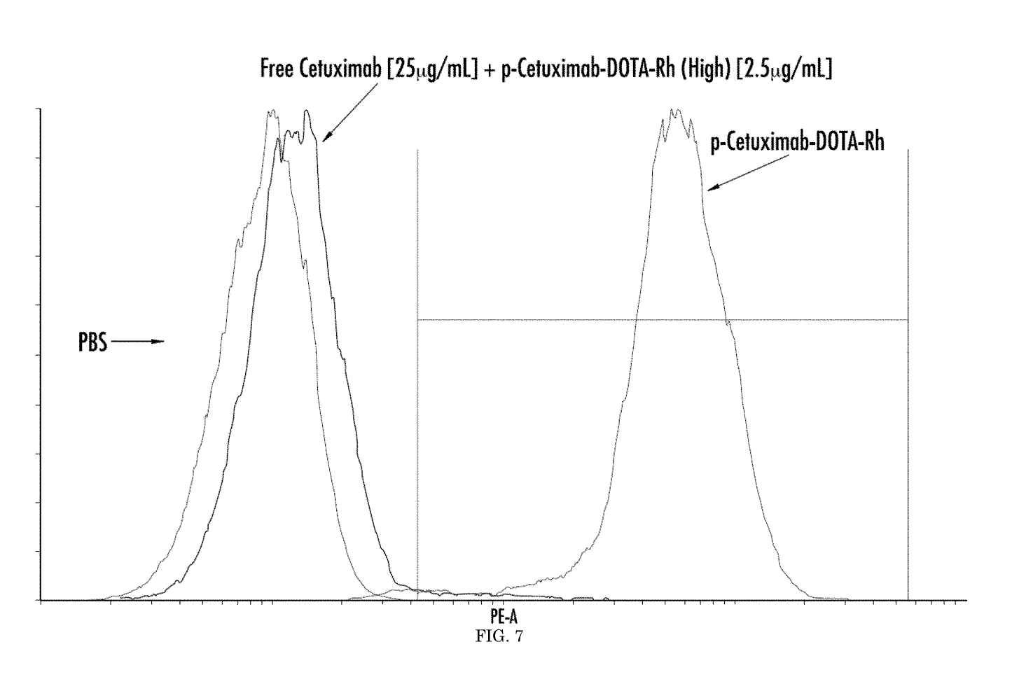

Specificity of Cetuximab to EGFR receptor was determined by Fluorescent Activated Cell Sorting (FACS) based on binding of Rhodamine-labelled Gd-DOTA-Polycefin-Cetuximab (2.5 μg/mL to EGFR expressed in MDA-MB-468 cells (amount 30,000) in comparison to that of phosphate buffered saline (PBS) (negative control) and free Cetuximab (positive control) (FIG. 7). This figure shows that the peak to the right corresponds to Rhodamine-labelled Gd-DOTA-Polycefin-Cetuximab bound to EGFR. In comparison, the peak the in the middle of histogram was found to correspond to free unlabeled Cetuximab at 25 μg/mL which did not bind EGFR. The positions of the peak corresponding to free unlabeled Cetuximab and the peak corresponding to that of the negative control PBS were very close.

Analysis data indicated that both anti-mouse TfR mAb and Cetuximab conjugated to Polycefin-Gd-DOTA preserved their functional activities and may be active during in vivo MRI.

### Example 9. Materials and Methods for Tumor-Type Specific MRI

Cell Lines and Culture Conditions.

Human breast cancer cell line MDA-MB-468 (TNBC, EGFR positive) and human lung cancer cell line A549 (EGFR positive) were obtained from American Type Culture Collection (Manassas, Va.). Cells were cultured in L-15 and F-12K medium, respectively, supplemented with 10% FBS and antibiotics/antimycotics.

Tumor xenografts in nude mice. All experiments with animals were performed in accordance with the protocols approved by the Cedars-Sinai Medical Center Institutional Animal Care and Use Committee (IACUC). Athymic NCr-nu/nu mice were obtained from NCI-Frederick. MDA-MB-468 cells were stereotactically implanted at either 1.5×10⁶ or 2.5×10⁶ into the right basal ganglia field of mice. A549 cells were stereotactically implanted at 5×10⁵.

Xenogen Fluorescent Imaging.

For the MRI and near infrared studies of contrast agent accumulation in the healthy brain and tumor tissue, the mice were anesthetized by inhalation of Isoflurane (2-4% to effect) inside an induction chamber. Once anesthetized, the mice were removed from the chamber; their tail was dipped in warm water to allow the vain to dilate and placed in a Mouse Tail Illuminator (Braintree Scientific Inc., Braintree, Mass.) to avoid failure of injection due to unexpected fast recovery from anesthesia. Contrast agent in PBS at a dose 0.1 mmol Gd/kg was administered via the tail vein of desired via the tail vein using a 30-gauge needle 1 ml syringe, at a rate of 100 μl within 5 seconds. (Single injection per mouse). Then, mice were anesthetized again by inhalation of Isoflurane (2-4% to effect) inside of an induction chamber before image detection. A nose cone was placed to maintain anesthesia during MRI measurements. During measurements, 1.8% isoflurane was maintained. The mouse bed was heated to prevent cooling of the mice during anesthesia.

MRI Measurement.

The MRI sessions were performed on a Siemens Microscan System 9.14 Tesla, 45 after (for A549) and 27, 48 and 52 days after (for MDAMB 468) cell inoculation when tumors were ˜4 mm in diameter. Spin echo and T1 images of the entire brain were acquired. Axial slices were positioned over the entire brain. A multisided echo sequence was used with TR=900 ms. 50 Slices with a 0.5 mm thickness were acquired for a 1.8×1.8 cm field of view with a 196×196 matrix size. The in-plane resolution was 92×92 μm/pixel. T1 values of the samples were measured from regions of interest using a single exponential fitting of the intensity for different repetition times scans. In this case, the in-plane resolution was 234×234 μm/pixel.

Xenogen IVIS 200 Imaging.

For the assessment of drug distribution and localization in nude mice, animals were studied in a Xenogen IVIS 200 imager under isoflurane anesthesia at different time points (before drug administration and 24 h after the injection of the drug). Twenty-four hours after drug administration, mice were euthanized. Intra-arterial PBS perfusion was done in order to wash out the circulating drugs in blood vessels. The tumor and major organs were harvested to detect the fluorescent signal. The fluorescent signal intensities in the tumor and different organs were analyzed by Xenogen Living ImageH software, Version 2.50 (WaveMetrix, USA).

### Example 10. MRI-Ennhancement by Gd-DOTA-Polycefin

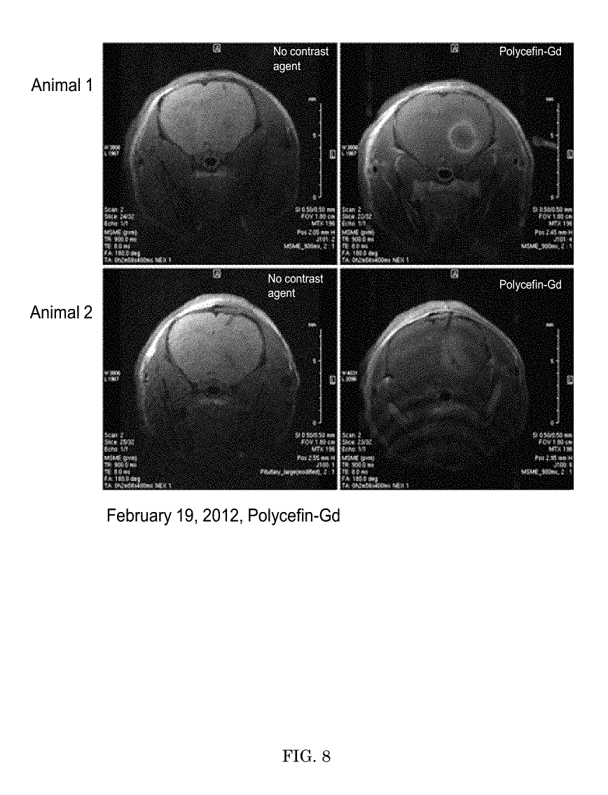

Initial experiments with TNBC tumor A549 were negative due to insufficient Gd-DOTA bound to polymalic acid in a Polycefin nanoconjugate (less than 5%), % refers to the fraction of total carboxyls of the polymalic acid platform covalently bound to Gd-DOTA. Subsequent experiments were conducted with Gd-DOTA-Polycefin loaded with 12-13% Gd. FIG. 8 shows the result of imaging of two animals representing mouse model of TNBC injected with the human TNBC-specific MRI enhancer nanoconjugate. MRI imaging of human TNBC on mouse was performed 27 days after tumor inoculation. Referring to FIG. 8, it was observed that animals injected with a Polycefin-Gd nanoconjugate displayed considerable accumulation of Polycefin-Gd in tumors which made tumors visible. In contrast, no tumors were visible on images of animals which were not injected with the contrast agent. The data shows feasibility of MR imaging using a Polycefin-Gd nanoconjugate.

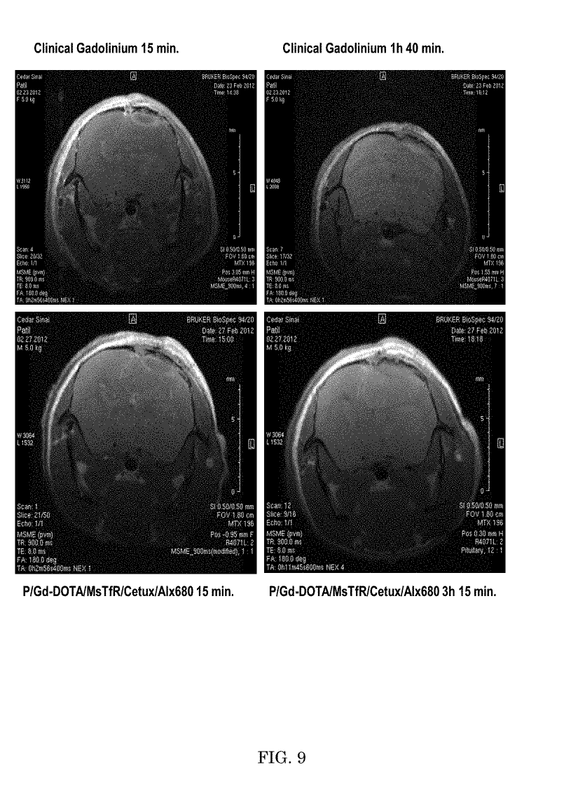

FIG. 9 shows MR imaging of the animals having the same type of tumors as shown in FIG. 8 using a Polycefin-Gd nanoconjugate and a commercially available Gd(III) enhancer reagent. However the time of injection of Gd(III) enhancer reagents were 49-52 days after tumor inoculation. This tumor MRI was used for to time dependent evaluation. Top images show administration of Gd(III) for clinical use. Top image on the left was made 15 minutes after reagent injection and shows visible tumor. Top image on the right was made 1 hour 40 minutes after injection of Gd (III) and does not show tumor image, because Gd(III) enhancer reagent was already cleared through the kidneys. Bottom images show administration of a Gd-DOTA-Polycefin nanoconjugate specific for EGFR expression on TNCB cells. Lower image on the left was made 15 minutes after injection of P/Gd-DOTA/MsTfR/Cetux/Alx680 nanoconjugate and shows visible tumor. Lower image on the right was made 3 hours 15 minutes after injection of the nanoconjugate. The data indicates that the enhancement effect of Gd-DOTA-Polycefin is retained for much longer time than that of the Gd(III) reagent routinely used in clinics. This prolongation may be explained by an effect of slower clearance through the kidneys as it takes longer to clear the nanoconjugate because of its high molecular weight above clearance cut-off, and retention of the polymer bound Gd(III) because of tumor specific binding.

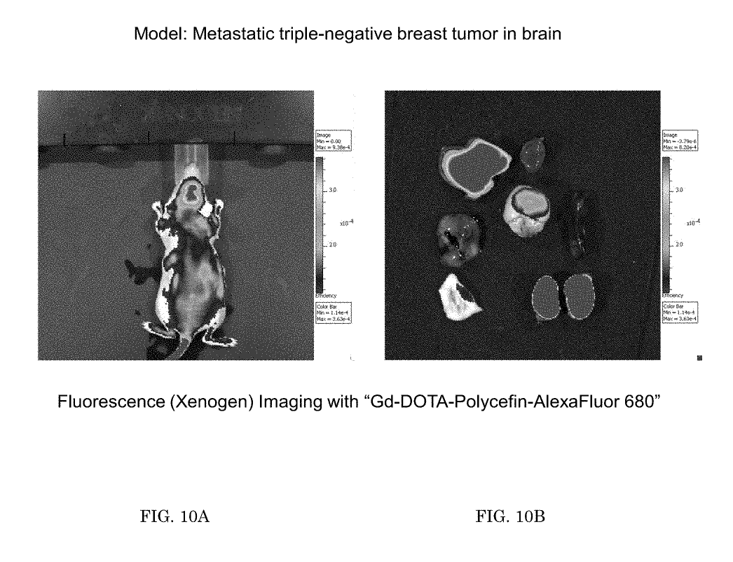

To evaluate specific localization of accumulated enhancement reagents within a body of an experimental animal, Alexa Fluor 680 was attached to a Gd-DOTA-Polycefin nanoconjugate for Xenogen imaging using fluorescence. Referring to FIGS. 10A-10B, the image on FIG. 10A demonstrates high amounts of imaging agent accumulated in kidneys and liver of an animal. The image on FIG. 10B shows tumor in the middle identifiable by blue fluorescence and accumulation of Polycefin-Gd-Alexa Fluor 680.

### Example 11. Evaluation of Specificity of MRI Enhancement Reagents

To separate the retention effect based on binding to target from the prolonged natural clearance effect through the kidneys the kinetics of the T1-values were evaluated.

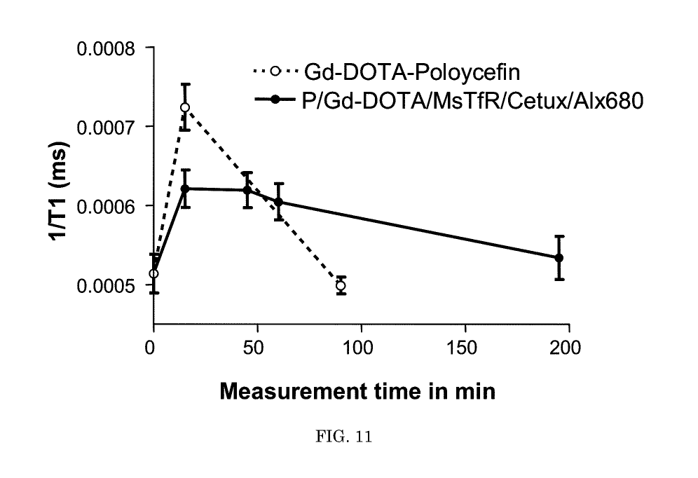

FIG. 11 shows kinetics of MR imaging after injecting the subject with clinically used formulation of Gd(III) and formulation of a Gd-DOTA-Polycefin nanoconjugate carrying covalently bound Cetuximab. The kinetics of imaging was not deconvoluted and contain effects of different blood clearance times due to different molecular weights and, in the case of Gd-DOTA-Polycefin retention by interaction of covalently bound Cetuximab with EGFR on tumor cell surface. Referring to FIG. 11, it was observed that the high values of 1/T1 were maintained for several hours for Polycefin-Gd-DOTA while the curve for clinical Gd(III) rapidly decayed after reaching a maximum value. The differences in kinetics profiles may be explained by the fact that the 1/T1 value depends on the amount of clinical Gd(III) or Polycefin bound Gd(III) in the circulating blood; and on the retention of Polycefin-Gd-DOTA by the tumor. Clinical Gd(III) cannot penetrate BBB and is not retain by the tumor, and may only circulate in the tumor blood capillaries. The levels of both a clinically used Gd(III) and Polycefin-Gd-DOTA decrease because of clearance through kidneys. However, the clearance of Polycefin-Gd-DOTA is slower than that of clinically used Gd(III) because large molecules, such as Polycefin-Gd-DOTA are less rapidly cleared.

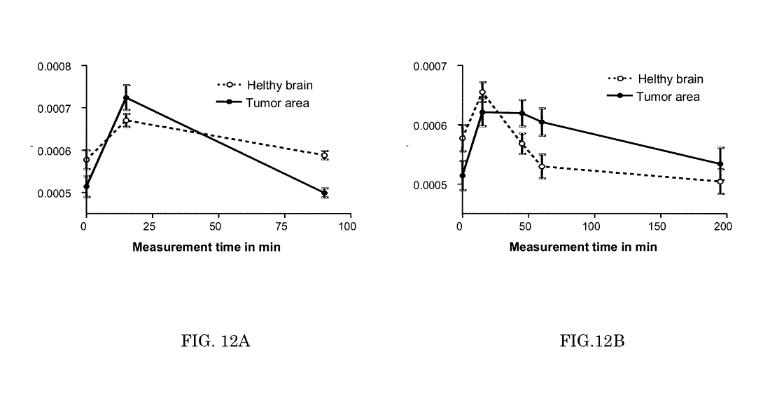

FIGS. 12A and 12B compares kinetics of T1 relaxation of MRI for healthy and tumor areas of brain after injection of clinically used Gd(III) enhancer reagent and a Gd-DOTA-Polycefin nanoconjugate containing Cetuximab. FIG. 12A shows that after application of Gd(III) 1/T1 values obtained for a healthy and tumor areas of brain are not significantly different for 50 minutes following the injection of the contrast agent. The data may be explained by the fact that the Gd(III) formulation does not recognize the tumor. FIG. 12B shows that 1/T1 values obtained for a healthy and tumor areas of brain after injection of the Gd-DOTA-Polycefin nanoconjugate containing Cetuximab are significantly different. Half-life of the nanoconjugate in the healthy area of brain is 20-30 minutes and that in the tumor area is 130 minutes. The higher half-time value obtained for the noconjugate in the tumor area may be explained by retention of the nanoconjugate by tumor due to specific binding of the nanoconjugate to EGFR.

### Example 12. MRI-Enhancing Reagents Targeting Different Types of Tumors

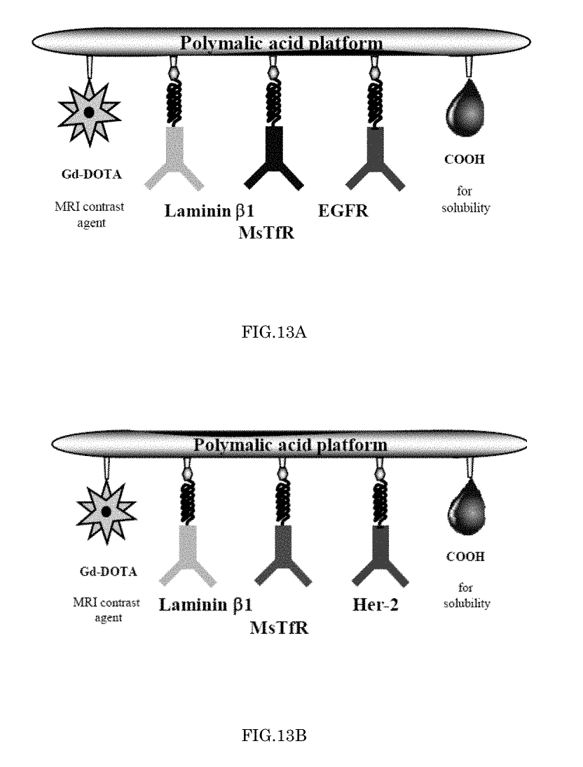

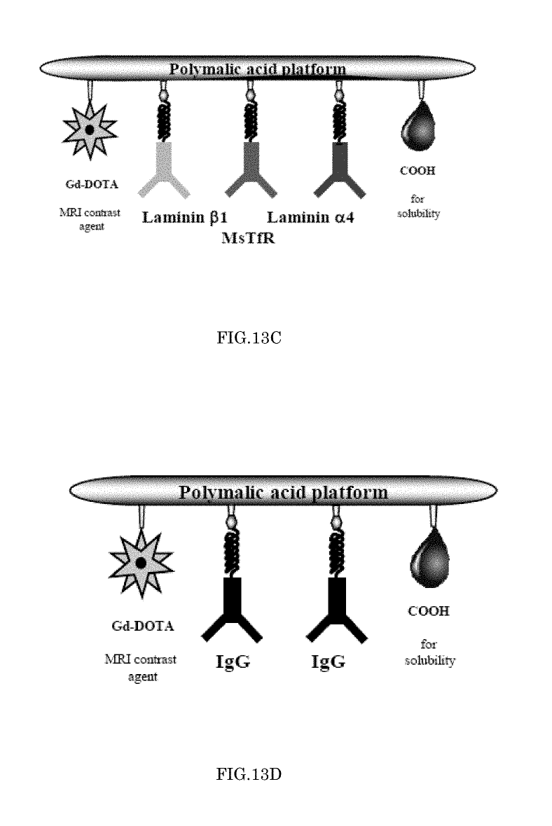

Imaging of different types of tumors involves formulation of nanoconjugates having an ability to target tumors specifically and differentiate between different types of tumors. FIGS. 13A-13D show schematic drawings of molecules designed to target primary brain and TNBC metastasized to brain (FIG. 13A), HER2-positive brain cancer metastasized to brain (FIG. 13B), glioblastoma (FIG. 3C) and a control molecule lacking specific targeting modules (FIG. 13D). All nanoconjugates of FIGS. 13A-13D were designed for targeting specific tumors and a control molecule include Gd-DOTA, as an MRI contrast agent for MRI, and having a carboxyl group COOH for improving solubility, where each of these moieties attached to polymalic acid platform. Referring to FIG. 13A, a nanoconjugate designed for targeting and imaging primary brain and metastatic brain tumor of triple negative breast cancer includes mAbs for targeting: mAb specific to laminin β1, MsTfR specific mAb and Cetuximab specific to EGFR. Referring to FIG. 13B, a nanoconjugate designed to target and facilitate imaging of HER2 positive breast cancer metastasized to brain includes mAbs for targeting: mAb specific to laminin β1, Herceptin® specific to HER2 and TfR mAb. Referring to FIG. 13C nanoconjugate designed to target and facilitate imaging of glioblastoma includes mAbs for targeting: mAb specific to laminin β1, mAb specific to laminin α4 and MsTfR specific mAb.

Referring to FIG. 13D, a nanoconjugate designed as a control for other agents includes mouse mAbs for targeting: two IgG1 monoclonal antibodies that do not bind specific targets in tumors.

Validation of specific effect of the nanoconjugates on MRI is performed on mouse models of TNBC, the HER2-positive breast cancer metastasized to brain and glioblastoma. These models may also be used to differentiate specific and non-specific effects of the nanoconjugates on MRI. For example, although the nanoconjugates are designed for binding to specific targets, unspecific penetration of the nanoconjugates may occur through the permissive (while damaged) endothelia of BBB called a typical tumor effect due to an “enhanced permeation and retention” (EPR). Although access to tumor tissue from the blood may be possible by the EPR effect, recognition of tumor specific cells in the described TNBC, HER2-positive-tumor and gioblastoma is restricted to EGFR, HER2 and laminin receptors, targeted recognition by antibodies against EGFR, HER2, and laminin-α4 and lamini-ß1. Images obtained after application of nanoconjugates designed for specific targets can be compared with control images. Together the results will indicate the strength of specificity and the “background” effect of injection of the control molecule, in which specific targeting modules are replaced with unspecific non-targeting IgG1 mAbs. The background effect is of interest for translation into the human system since in human tumors, transferrin receptors are typically present in capillary endothelia and on tumor cell surface. A tumor specificity may be improved by eliminating the anti-human TfR mAb and relying only on the EPR effect for penetration of BBB and targeting cancerous tissues.

For determining a tumor type, the nanoconjugates containing specific targeting modules can be administered following the injection with the non-specific MRI agents, such as gadobenate dimeglumine (MultiHance®, Gd-BOPTA), or any other tumor-type unspecific agents which penetrate the tumor BBB (Uggeri, F et al., 1995, Novel Contrast Agents for Magnetic-Resonance-Imaging—Synthesis and Characterization of the Ligand Bopta and Its Ln(Iii) Complexes (Ln=Gd, La, Lu)—X-Ray Structure of Disodium (Tps-9-145337286-C-S)-[4-Carboxy-5,8,11-Tris(Carboxymethyl)-1-Phenyl-2-Oxa-5,8,11-Triazatridecan-13-Oato(5-)]Gadolinate (2-) in a Mixture with Its Enantiomer, Inorganic Chemistry, 34 (3), 633-642, which is incorporated herein by reference as if fully set forth).

The presence of cancer can be determined by obtaining a first MRI image of the tissue in the brain following the administration of the non-specific contrast agent. Measuring the intensity of the MRI signal in the first image indicates the presence of a tumor. If no signal is found, the brain does not contain a tumor. If tumor is detected in the first MRI image, the location and intensity of the this first MRI is compared with the location and intensity of an image obtained from a second MRI after injecting a contrast agent that contains a targeting molecule that identifies the tumor type by reaction of the specific antibody, peptide or other specific agent.

The second contrast agent generates the location and intensity of the second MRI image only if this contrast agent recognizes the type of the tumor seen already in the MRI picture identified by the tumor-type nonspecific contrast agent MultiHance® in the first MRI. The identity is indicated by the exact co-localization of this (first) MRI picture with the location of the tumor signal obtained in the second picture generated after injection of the contrast agent that is tumor-type specific targeting, e.g., glioblastoma or HER2 positive tumor or a family of tumors. This second injection using the tumor variant specific contrast agent has to be applied no earlier than 2-3 hours after the injection of MultiHance® in order not to interfere with the MRI image generated by the contrast agent MultiHance®. The MRI images can be obtained at 30 min, 60 min, 180 min and 360 min after the injection of the particular contrast agents. The intensities of the MRI images will vary with the time elapsed after injection. Overall, the dynamics of the signals are different in the time series of the first MRI performed with the contrast agent MultiHance® and the second MRI performed with the type-specific contrast agent: the MultiHance® MIR intensities fade faster than the MRI intensities for the tumor specific contrast agents.

The intensities have to be compared at identical time points after subtraction of background intensities measured for regions of interest (ROIs) outside of the tumor region but otherwise in similar locations in the brain. Typically, MRI images include scans of both brain hemispheres. If one hemisphere does not contain tumor, this hemisphere can be used for selecting ROIs for background intensities in appropriate locations. Then, the measured intensity for a given ROI is divided by the area spanning the ROI. The ratios obtained for background ROI are used to correct ROI of obtained for the tumors in the picture of the first and second MRI. For the correction, the background is subtracted from the MultiHance® generated image and tumor-type ROIs.

After obtaining the corrected and averaged ROIs value for selected regions which may encompass part or whole tumor, the corrected (averaged) ROI for the MultiHance® MRI is subtracted from the ROI of the MRI obtained from the tumor-specific (targeted) contrast agent. The difference should be significantly greater than zero at the times 180 min to 360 min after injection if a specific tumor is indicated. The identity of the MRI signal with a given tumor is also verified by the “co-localization” of the image obtained in the first MRI (using MultiHance® as the contrast agent) at 30 min and the image in the second MRI (tumor-type specific contrast agent) obtained after 180 min and/or 360 min after injection of contrast agent. The results at ambient times validate or disprove the identification.

The picture obtained by the first MRI after injection of the first (MultiHance®) has the purpose to identify a tumor but in a non-specific manner. If the image after injection of the second MRI contrast agent, for example, the nanoconjugate that carries the anti-EGFR antibody Cetuximab as shown on FIG. 13A, is empty, the result shows the absence of glioblastoma, TNBC (triple negative breast metastatic cancer), or lung metastatic tumor. In this case, the second MRI is repeated following 2-3 hours after injection of the previous tumor-type specific contrast agent by injecting the contrast agent targeting HER2, for example, the nanoconjugate that carries the anti-HER2 antibody, Trastuzumab (Herceptin) as shown on FIG. 13B. If the subsequent MRI image results in a visible signal sufficing the above identification criteria, it is indicative of the presence of the HER2-positive metastatic breast cancer.

In case the MRI after injection of the contrast agent targeting specifically EGFR (FIG. 13A) is positive, a following MRI should be performed because Cetuximab (the EGFR-specific antibody) indicates a family of tumors carrying the EGFR antigen. For this purpose, the patient in the subsequent MRI can be injected with a contrast agent carrying antibodies recognizing laminin 411 which recognize glioblastoma, for example, as shown on FIG. 13C. Another contrast agent may carry anti-insulin antibody recognizing a brain tumor overexpressing insulin receptor. Still another contrast agent, which targets tumor necrosis factor can recognize the first MRI signal as positive for inflammation.

An abbreviated method can be accomplished by merely applying the second MRI agent as a single means for diagnosis of HER2-positive breast cancer or using a contrast agent with highly tumor-type specific targeting antibody, polypeptide, peptide, RNA of any other highly specific targeting tumor-type device. This is possible employing the fading kinetics, i.e., carrying out the second MRI in a time period of 180 minutes to 360 minutes after injection of the particular contrast agent. However, the fading kinetics have to be established under strictly reproducible synthesis of the contrast agents and application of validated injection dosages when used resulting in significant MRI signal at 180-360 minutes after injection under conditions of positive tumor recognition. This was accomplished for targeting HER2 antigens (FIG. 13B) or EGFR antigens (FIG. 13A).

### Example 13. MRI Enhancing Reagent Targeting Alzheimer Plaques

An MRI enhancing nanoconjugate was designed to image Alzheimer plaques. Previously it was shown that curcumin can bind beta amyloid plaque (Ryu E K et al. 2006 J Med Chem 49: 6111).

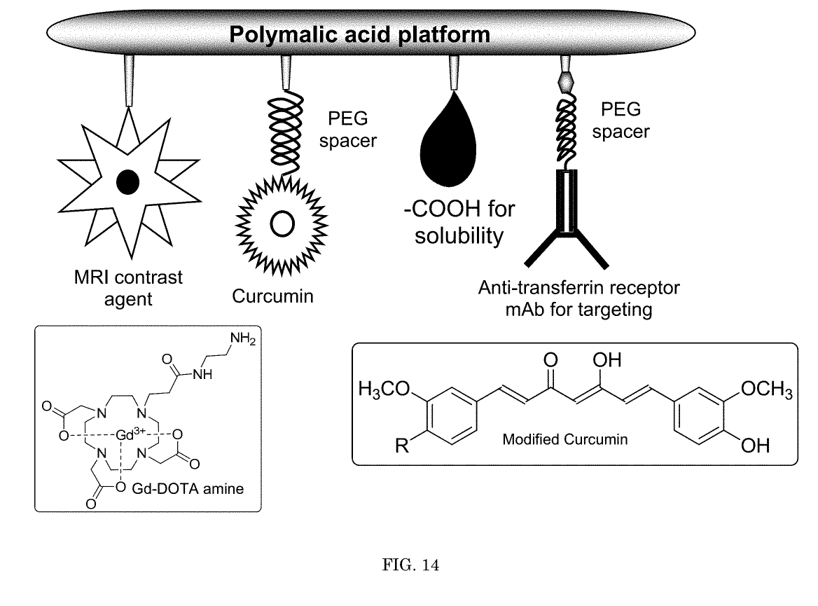

A nanoconjugate based on polymalic acid contains simultaneously attached curcumin (5-hydroxy-1,7-bis(4-hydroxy-3-methoxyphenyl)-1,4,6-heptatrien-3-on) and Gd-DOTA (2,2′,2″-(2-(2-(2mercaptoethylamino)-2-oxoethyl)-1,4,7-tetraazacyclododecane-1,4,7-triyl)triacetic acid), and is designed to target and image Alzheimer's disease beta-amyloid plaques in vivo (FIG. 14). The nanoconjugate is a composite molecule containing features of Polycefin and the following chemically functional modules: an MRI contrast agent Gd-DOTA, curcumin for binding amyloid plaques, a carboxy group and TfR specific mAb attached to polymalic acid. Each of curcumin and TfR mAb modules is linked to polymalic acid by the PEG spacer.

To study in vivo imaging in mouse and rat models of Alzheimer's disease (AD models), mouse or ratTfR mAb could be used. Mouse or rat TfR could be replaced with human TfR for imaging in human patients. A nanoconjugate can carry multiple curcumin molecules which may result in firm attachment of a nanoconjugate around beta-amyloid plaque contributing to sharp contours with high contrast. Nanoconjugate molecules containing curcumin can also carry a large number of covalently attached Gd-DOTA, typically 40-60 or more Gd per molecule of nanoconjugate. This high concentration of Gd on amyloid plaques may allow imaging by MRI at high contrast and resolution quality. The optional covalent attachment of a tracking dye may facilitate gross in vivo monitoring of the nanoconjugate distribution by Xenogen imaging systems other than by using MRI and may allow the validation of curcumin-Polycefin (Gadolinium absent) entrance into brain in the first phase of the synthesis/investigation. A tracking dye may also validate whether curcumin is attached to Polycefin within the brain. Thus, it can be useful in optimization experiments with curcumin-Polycefin in the absence of Gd.

If curcumin binding is not sufficiently strong, an antibody that specifically recognizes human Alzheimer plaques may be used. Penetration of the enhancement reagent may be accomplished by the attached anti-transferrin antibody (anti-TfR mAb), which carries the enhancer through the BBB by transcytosis. Because BBB transcytosis is reversible, the enhancer reagent could be very firmly attached to the plaques. If curcumin does not bind sufficiently strong, the plaque-specific mAb may be attached to the platform instead of curcumin. Multiple curcumin residues attached to the platform may enhance the strength of plaque binding through multiple binding. If further strength enhancement is designed an alternative of using the antibody may be employed.

### Example 14. General Procedure for N-Alkylation

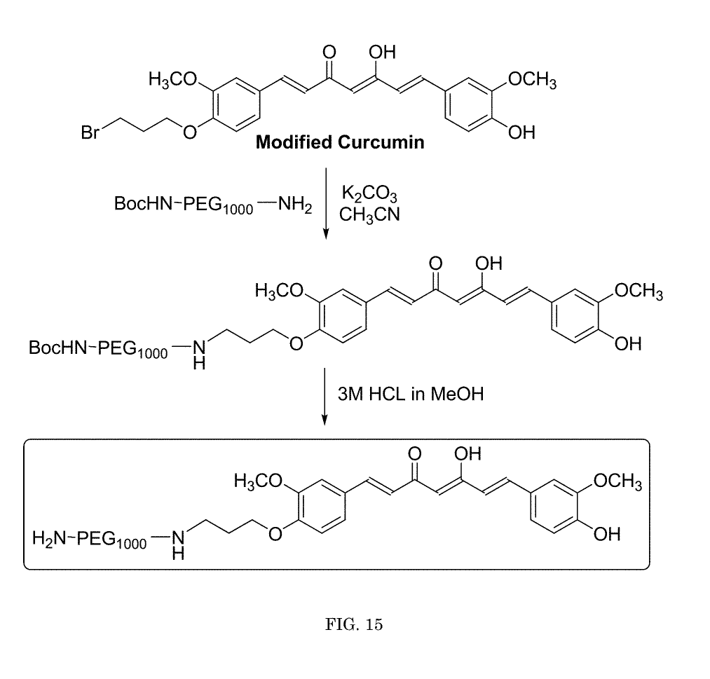

FIG. 15 shows synthesis of a curcumin-PEG₁₀₀₀-amine. A solution of Boc-PEG₁₀₀₀-NH₂ (0.2 mmol) in 2 ml of acetonitrile was added to a suspension of K₂CO₃ (1.2 mmol) in 2 ml acetonitrile, and the reaction mixture was stirred at room temperature for 10 min. A solution of modified curcumin (0.2 mmol) in 2 ml acetonitrile was added to the reaction mixture, and the reaction was allowed to proceed at RT for 72 hours. The reaction mixture was filtered to remove undissolved solids and washed with acetonitrile. The filtrate was concentrated and the residue was passed over sephadex LH 20 in methanol. Product containing fractions were collected, methanol was removed. Product was used for next step without further purification. Reaction yield was 73%.

### Example 15. General Procedure for Boc Deprotection

3M methanolic HCL 9 ml was added to Boc-NH-PEG₁₀₀₀-curcumin and reaction mixture was stirred at room temperature for 16 hour. Solvent was evaporated to dryness with rotary evaporator. Thick solid was dissolved in water and freeze dried to obtain a desired product as dark yellow solid. Reaction yield 96%.

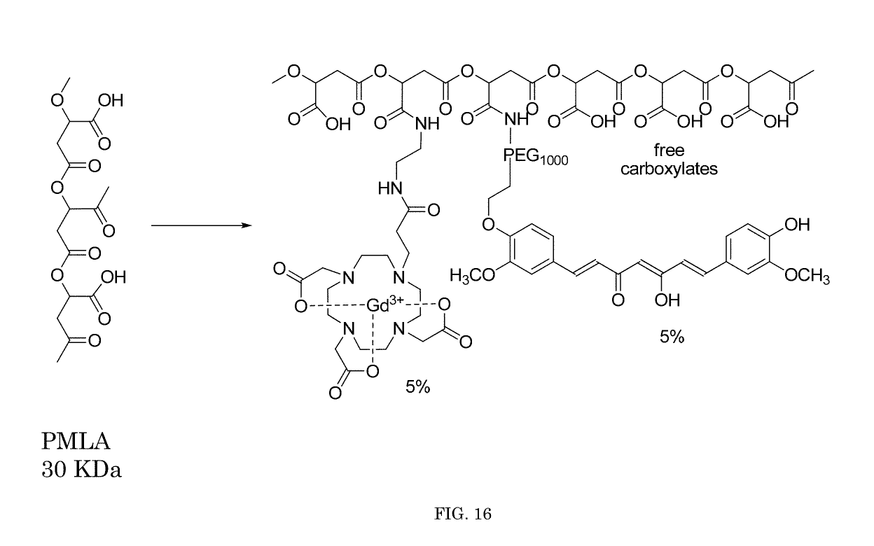

The curcumin derivatives will be covalently attached to NHS-activated carboxyls of polymalic acid together with 2-mercapto-1-ethylamin and Gd-DOTA to receive the MRI-enhancer as shown in FIG. 16. In this figure, curcumin and Gd-DOTA are shown to be attached to polymalic acid (PMLA, 30 KDa). Each of curcumin and Gd-DOTA is attached to 5% of polymalic acid pendant carboxylates. The percentage of attached modules may be increased up to 30% or more of pendant carboxylates to improve MRI enhancement of the reagent.

### Example 15. Binding of Polymalic Acid-Bound Curcumin (5%)

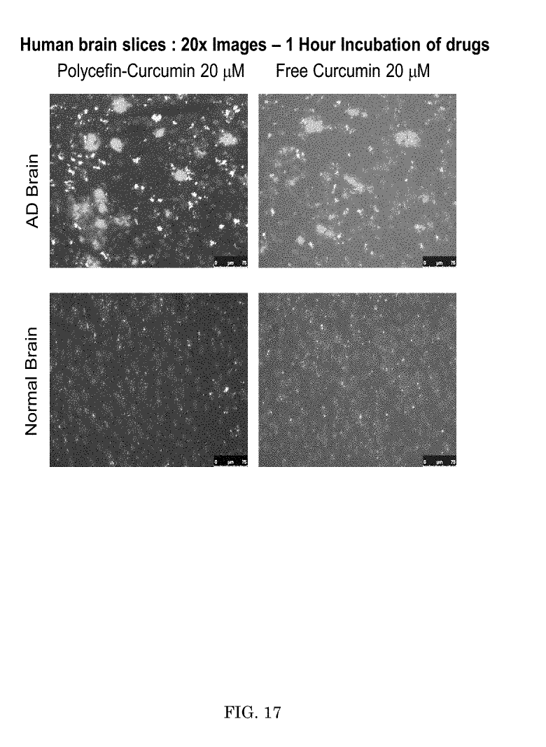

Polymalic acid-bound curcumin can be used to stain plaques in human brain tissue ex vivo (FIG. 17). Slices of human brains having AD (top images) and normal human brains (lower images) were analyzed by fluorescent imaging after staining with 20 μM of Polycefin-curcumin (images to the right) and 20 μM of free curcumin (images to the left). Referring to FIG. 17, the higher number of bright light spots observed on the top left image compared to that on the top right image indicates stronger binding of the polymalic acid-curcumin conjugate than of free curcumin to human Alzheimer plaques in slices of brain obtained from a patient having Alzheimer disease (AD). No binding occurred in control that included slices of brain obtained from a healthy individual as visible on the lower images. Concentration of a polymalic acid curcumin conjugate may be reduced to 2 μM. The use of polymalic acid-curcumin is advantageous compared to the used of free curcumin because it does not show staining background even at high concentrations, such as higher than 200 μM. This demonstrates that binding to polymalic acid greatly enhances the solubility of curcumin.

### Example 16. Diagnosing and Monitoring Alzheimer's Disease

The nanoconjugate Gd-DOTA/polymalic acid/Curcumin (5%)/anti-mouse TfR mAb may be used as MRI enhancer for imaging the plaques. The strategy for imaging may also include replacing of Gd-DOTA by the highly fluorescent dye AlexaFluor 680 and finding conditions that allow detection of fluorescence in the brain of Alzheimer-mouse using Xenogen Imaging System. Curcumin may also be replaced by anti-plaque mAb. For detection by fluorescence, the MRI system using Gd-DOTA (highest possible %)/polymalic acid/Curcumin or anti-plaque antibody/anti-TfR mAb for imaging.

The references cited throughout this application, are incorporated for all purposes apparent herein and in the references themselves as if each reference was fully set forth. For the sake of presentation, specific ones of these references are cited at particular locations herein. A citation of a reference at a particular location indicates a manner(s) in which the teachings of the reference are incorporated. However, a citation of a reference at a particular location does not limit the manner in which all of the teachings of the cited reference are incorporated for all purposes.

It is understood, therefore, that this invention is not limited to the particular embodiments disclosed, but is intended to cover all modifications which are within the spirit and scope of the invention as defined by the appended claims; the above description; and/or shown in the attached drawings.

1. A method of imaging a cell or a tissue in a brain of a subject and detecting cancer comprising:
co-administering to a subject a contrast agent and a composition comprising a polymalic acid-based molecular scaffold conjugated to at least one imaging moiety, and at least one targeting module, wherein the imaging moiety comprises a chelating molecule and a paramagnetic ion chelated by the chelating molecule; the targeting module is independently selected from the group consisting of: an antibody, a peptide, a polypeptide, an oligonucleotide, and a therapeutic chemical; and co-administering comprises sequential administering of the contrast agent and the composition;
visualizing the contrast agent and the imaging moiety in the brain of the subject by an MRI technique;
detecting the presence of a cancer in the brain of the subject, wherein detecting comprises obtaining a first MRI image of the brain after administering the contrast agent; obtaining a second MRI image of the brain after administering the composition; measuring the intensity of an MRI signal in the first image and the second image; and comparing the intensity and location of the MRI signal in the first image with the intensity and location of the MRI signal in the second MRI image, wherein similarity in the intensity and location of the MRI signal in the first MRI image relative to the second MRI image is indicative of the presence of the cancer in the brain of the subject.

2. The method of claim 1, wherein the contrast agent is a Gd-based contrast agent selected from the group consisting of: gadoterate, gadodiamide, gadobenate, gadopentetate, gadoteridol, gadoversetamide, and gadobutrol.

3. The method of claim 1, wherein the chelating molecule is selected from the group consisting of: 1,4,7,10-tetraazocyclododecane-1,4,7,10-tetraacetic acid, diethylenetriaminepentaacetic acid, 1,4,7,10-tetraazacydododecane-1,4,7,10-tetrakis(2-propionic acid), and 1,4,8,11-tetrazacyclotetradecane-1,4,8,11-tetraacetic acid.

4. The method of claim 1, wherein the paramagnetic ion is selected from the group consisting of: gadolinium, chromium, manganese, iron, dysprosium, europium, and terbium.

5. The method of claim 1, wherein the contrast agent comprises Gd-DOTA.

6. The method of claim 1 wherein the targeting module is an antibody that specifically binds to a protein selected from the group consisting of: an epidermal growth factor receptor, human epidermal growth factor receptor 2, laminin 411, insulin-like growth factor, transferrin receptor protein, and tumor necrosis factor-alpha.

7. The method of claim 6, wherein the antibody comprises at least one of Cetuximab or Trastuzumab.

8. The method of claim 1, wherein the composition comprises more than one targeting modules.

9. The method of claim 8, wherein more than one targeting modules comprise an antibody that specifically binds the transferrin receptor protein.

10. The method of claim 1, wherein the cancer is a primary brain tumor or a metastatic brain tumor.

11. The method of claim 10, wherein the primary brain tumor is a glioblastoma.

12. The method of claim 10, wherein the metastatic brain tumor is selected from triple negative breast cancer metastasized to the brain, HER2-positive breast cancer metastasized to the brain, and lung cancer metastasized to the brain.

13. The method of claim 1, wherein the targeting module comprises Trastuzumab, and the similarity in the intensity and location of the MRI signal in the first MRI image relative to the second MRI image is indicative of the HER2-positive breast cancer metastasized to the brain.

14. The method of claim 1, wherein the targeting module comprises Cetuximab, and the similarity in the intensity and location of the MRI signal in the first MRI image relative to the second MRI image is indicative of an EGFR-expressing tumor.

15. The method of claim 14, wherein the EGFR-expressing tumor is a primary cancer or a metastatic cancer, wherein the primary cancer is glioblastoma and the metastatic brain cancer is a triple negative breast cancer metastasized to the brain, or lung cancer metastasized to the brain.

16. The method of claim 15, further comprising differentially diagnosing the glioblastoma from the metastatic EGFR-expressing tumors by administering to the subject a composition comprising a polymalic acid-based molecular scaffold conjugated to at least one imaging moiety, and a targeting module comprising an antibody that specifically binds to laminin 411; obtaining a third MRI image of the same location in the brain after administering the composition comprising the antibody that specifically binds to laminin 411; measuring the intensity of an MRI signal in the second image and the third image; and comparing the intensity and location of the MRI signal in the second image with the intensity and location of the MRI signal in the third MRI image, wherein similarity in the intensity and location of the MRI signal in the second MRI image relative to the third MRI image is indicative of the presence of the glioblastoma in the brain of the subject.

17. The method of claim 16, wherein the sequential administration includes an administration of the composition in a time period of 2 hours to 3 hours subsequent to the administration of the contrast agent.

18. The method of claim 17, wherein the intensity of the MRI signal in the first image is measured at least 30 minutes subsequent to the administration of the contrast agent.

19. The method of claim 18, wherein the intensity of the MRI signal in the second or third image is measured at a time of 40 minutes to 360 minutes subsequent to the administration of the composition comprising the targeting module.
